(19) United States
(12) Patent Application Publication
(10) Pub. No.: US 2026/0232846 A1
(43) Pub. Date: Aug. 13, 2026

(54) FLUORESCENT PROBE COMPOUND AND COMPOSITE COMPRISING SAME

(71) Applicant: KOREA RESEARCH INSTITUTE OF BIOSCIENCE AND BIOTECHNOLOGY, Daejeon (KR)

(72) Inventors: Chang-Soo LEE, Daejeon (KR); Kyungkwan LEE, Daejeon (KR); Chul Soon PARK, Daejeon (KR)

(73) Assignee: KOREA RESEARCH INSTITUTE OF BIOSCIENCE AND BIOTECHNOLOGY, Daejeon (KR)

(21) Appl. No.: 18/998,003

(22) PCT Filed: Jul. 21, 2023
(86) PCT No.: PCT/KR2023/010540

§ 371 Date: Dec. 4, 2025

(30) Foreign Application Priority Data
Jul. 25, 2022 (KR) 10-2022-0091943

(51) Int. Cl. A61K 49/00; C09K 11/06

(52) U.S. Cl. CPC A61K 49/0021; C09K 11/06; C09K 2211/1011; C09K 2211/1018; C09K 2211/1022

(57) ABSTRACT

The present invention relates to: a fluorescent probe compound represented by chemical formula 1 or chemical formula 2; and a composite comprising bioceramics, and a fluorescent probe compound represented by chemical formula 1 and/or a fluorescent probe compound represented by chemical formula 2, which are bound to the bioceramics.

### CROSS-REFERENCE TO RELATED APPLICATIONS

This application is a National Stage of International Application No. PCT/KR2023/010540 filed on Jul. 21, 2023, claiming priority based on Korean Patent Application No. 10-2022-0091943, filed on Jul. 25, 2022, the disclosure of which is incorporated herein by reference in its entirety.

### TECHNICAL FIELD

The present invention relates to a fluorescent probe compound that exhibits fluorescence by specifically reacting to a bone-forming activity marker and/or a bone-resorbing activity marker, and a composite containing the same.

### BACKGROUND ART

Bones, which support a human body, are composed of organic components such as collagen and inorganic components such as calcium and phosphoric acid, the hard inorganic components increase compressive strength to withstand weight, and the organic components such as collagen increase elasticity and tension to withstand external pressure. In addition to supporting the body, bones serve as levers when muscles contract, and not only protect internal organs such as the brain and internal organs, but also store minerals such as phosphorus and calcium and are responsible for hematopoiesis, which involves producing blood in the bone marrow.

As described above, bones, which play a significantly important role in the human body, are regulated by the balance of osteoblasts and osteoclasts, which are involved in bone formation and bone resorption. Osteoblasts are cells that create osteocytes, are also called osteoblastocytus, and are the main cells of the skeletal system that synthesize bone matrix and deposit substances necessary for bone, such as Ca and Mg ions, on the bone to control the calcification of bone tissue. The osteoblast contains a developed granular endoplasmic reticulum in the cytoplasm close to the bone surface, Golgi apparatus located around the nucleus, mitochondria present in the cytoplasm outside the Golgi apparatus, and alkaline phosphatase (ALP), which is a glycoprotein enzyme, in the cell membrane.

Meanwhile, the osteoclasts are multinucleated giant cells that dissolve bone tissue, and function to dissolve bone when calcium in the blood is insufficient and bone calcium needs to be replenished, when bone has microscopic cracks or flaws, or when old bone needs to be replaced with new bone. Such osteoclasts are hematopoietic cells of the monocyte/macrophage lineage that originate from the bone marrow, and osteoclast precursors differentiate and develop into osteoclasts by growth factors and cytokines produced in the bone marrow, which play a role in destroying and absorbing bone.

However, when a disaster or disease occurs, such as bone damage caused by external impact or osteoporosis due to an imbalance between osteoblasts and osteoclasts, treatment is significantly difficult and it takes a considerable recovery period to repair the damage. In particular, in a case where a bone density is low or bone damage is severe, bone grafting or bone filling may be necessary for treatment. Accordingly, bone filling or bone grafting technologies such as autograft, allograft, xenograft, and natural/synthetic biomaterials have been developed.

However, techniques such as dual energy x-ray absorptiometry (DEXA) and micro computed tomography (Micro-CT) to check osteogenesis after bone filling or bone grafting are expensive, time-consuming, and difficult to predict the prognosis of osteogenesis.

Alkaline phosphatase (ALP) is a family of isoenzymes widely distributed in mammalian tissue and is used as a diagnostic indicator for various human bone-associated diseases and liver-associated diseases. In particular, since the activity of ALP increases when osteoblasts differentiate, ALP as described above may serve as a biomarker for detecting the activity/differentiation of osteoblasts. In addition, osteoclasts release hydrogen ions outside the cells when activated, and the resulting pH change may be used as a biomarker.

Therefore, there is a need to develop an appropriate monitoring system capable of detecting the activity of osteoblasts and/or osteoclasts by tracking ALP activity or pH change in a biological system in real time.

### DISCLOSURE OF THE INVENTION

### Technical Problem

An object of the present invention is to provide a fluorescent probe compound that generates fluorescent signals in different emission wavelength regions for detecting or sensing the activity of osteoblasts and/or osteoclasts.

Another object of the present invention is to provide a composite capable of real-time fluorescence monitoring of metabolic processes of bone formation and bone resorption inside a body by binding the fluorescent probe compound to bioceramics.

### Technical Solution

In order to achieve the above objects, an aspect of the present invention provides a fluorescent probe compound represented by the following Chemical Formula 1 or Chemical Formula 2:

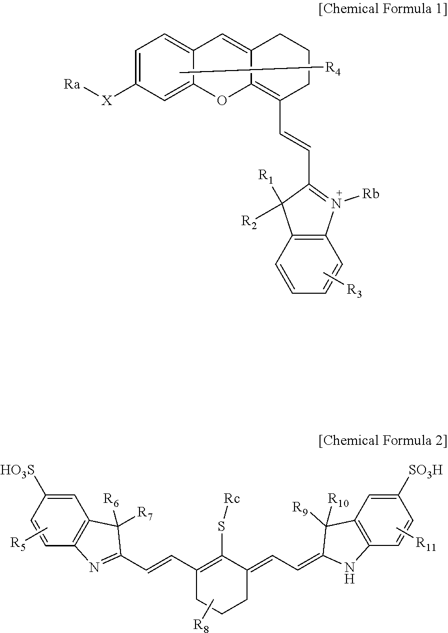

in Chemical Formula 1 and Chemical Formula 2,
X is O or NH,
R₁ to R₁₁ are the same as or different from each other, and are each hydrogen, deuterium, a halogen group, a hydroxy group, an amine group, a carboxyl group, a substituted or unsubstituted alkyl group having 1 to 10 carbon atoms, a substituted or unsubstituted cycloalkyl group having 3 to 10 carbon atoms, or a substituted or unsubstituted aryl group having 6 to 20 carbon atoms, and
Ra is the following Chemical Formula A, Rb is the following Chemical Formula B, and Rc is the following Chemical Formula C:

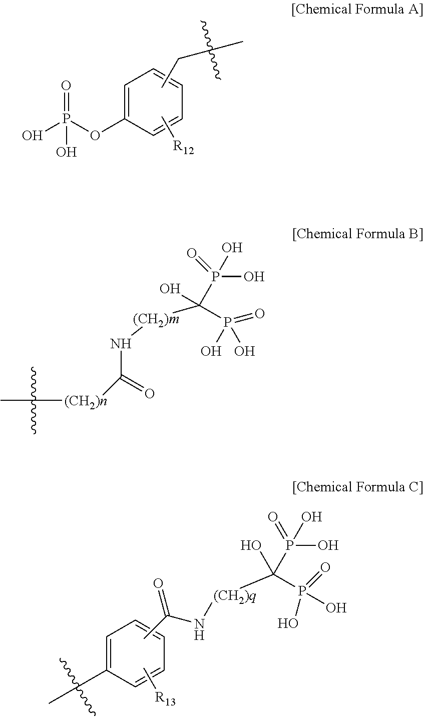

in Chemical Formula A to Chemical Formula C,
R₁₂ and R₁₃ are the same as or different from each other, and are each hydrogen, deuterium, a halogen group, a hydroxy group, an amine group, a carboxyl group, a substituted or unsubstituted alkyl group having 1 to 10 carbon atoms, a substituted or unsubstituted cycloalkyl group having 3 to 10 carbon atoms, or a substituted or unsubstituted aryl group having 6 to 20 carbon atoms, and
n, m, and q are a repeat number of a unit in parentheses, are the same as or different from each other, and are each 0 to 10.

In addition, another aspect of the present invention provides a composite containing bioceramics; and at least one selected from a bone-forming activity marker-sensitive fluorescent probe compound represented by Chemical Formula 1 and a bone-resorbing activity marker-sensitive fluorescent probe compound represented by Chemical Formula 2 that bind to the bioceramics.

In addition, still another aspect of the present invention provides a sensor containing the composite described above.

In addition, still another aspect of the present invention provides a composition for detecting a bone-forming activity marker containing a fluorescent probe compound represented by Chemical Formula 1.

In addition, still another aspect of the present invention provides a composition for detecting a bone-resorbing activity marker containing a fluorescent probe compound represented by Chemical Formula 2.

### Advantageous Effects

The present invention provides the fluorescent probe compound that reacts specifically to a bone-forming activity marker and exhibits fluorescence, and the fluorescent probe compound that reacts specifically to a bone-resorbing activity marker and exhibits fluorescence, such that bone metabolism processes and aspects may be detected in real time during a bone grafting 4 bone filling process without expensive equipment such as DEXA or Micro-CT.

In particular, when using a composite containing bioceramics to which a bone-forming activity marker-sensitive fluorescent probe compound and a bone-resorbing activity marker-sensitive fluorescent probe compound bind simultaneously, ALP according to osteoblast activity and pH change according to osteoclast activity are detected in different emission wavelength ranges, and fluorescent signals are emitted during bone grafting or bone filling; thus, it is possible to confirm bone remodeling, which enables diagnosis at the same time as grafting.

### BRIEF DESCRIPTION OF THE DRAWINGS

FIG. 1 is a diagram showing a 1H NMR spectrum of a bone-forming activity marker-sensitive fluorescent probe compound prepared in Synthesis Example 1 of the present invention.

FIG. 2 is a diagram showing a mass spectrometry spectrum of the bone-forming activity marker-sensitive fluorescent probe compound prepared in Synthesis Example 1 of the present invention.

FIG. 3 is a diagram showing a 1H NMR spectrum of a bone-resorbing activity marker-sensitive fluorescent probe compound prepared in Synthesis Example 2 of the present invention.

FIG. 4 is a diagram showing a mass spectrometry spectrum of the bone-resorbing activity marker-sensitive fluorescent probe compound prepared in Synthesis Example 2 of the present invention.

FIG. 5 is a diagram showing fluorescence images according to Experimental Example 1 of the present invention. In FIG. 5, NIR-OBL represents a composite A prepared in Example 1, NIR-OCL represents a composite B prepared in Example 2, and Co-labeled represents a composite C prepared in Example 3.

FIGS. 6A to 6C are diagrams showing fluorescence images according to Experimental Example 2 of the present invention.

FIGS. 7A to 7C are diagrams showing fluorescence images according to Experimental Example 3 of the present invention.

FIGS. 7D and 7E are diagrams showing SEM images according to Experimental Example 4 of the present invention.

FIGS. 8A to 8D are diagrams showing a result of a cytotoxicity test according to Experimental Example 5 of the present invention. In FIGS. 8A to 8D, NIR-OBL represents the composite A prepared in Example 1, NIR-OCL represents the composite B prepared in Example 2, and Co-labeled represents the composite C prepared in Example 3.

FIGS. 9A to 9D are diagrams showing a result of a cytotoxicity test according to Experimental Example 6 of the present invention. In FIGS. 9A to 9D, NIR-probe-co-labeled-CDHA represents the composite C prepared in Example 3.

FIG. 10 is a diagram showing fluorescence images according to Experimental Example 7 of the present invention. In FIG. 10, NIR-OBL-CDHA represents the composite A prepared in Example 1, and NIR-OCL-CDHA represents the composite B prepared in Example 2.

FIG. 11 is a diagram showing fluorescence images according to Experimental Example 8 of the present invention. In FIG. 11, NIR-OBL represents the composite A prepared in Example 1, NIR-OCL represents the composite B prepared in Example 2, and Co-labeled represents the composite C prepared in Example 3.

FIGS. 12A and 12B are diagrams showing fluorescence efficiency according to Experimental Example 8 of the present invention. In FIGS. 12A and 12B, NIR-OBL-CDHA represents the composite A prepared in Example 1, NIR-OCL-CDHA represents the composite B prepared in Example 2, and Co-labeled-CDHA represents the composite C prepared in Example 3.

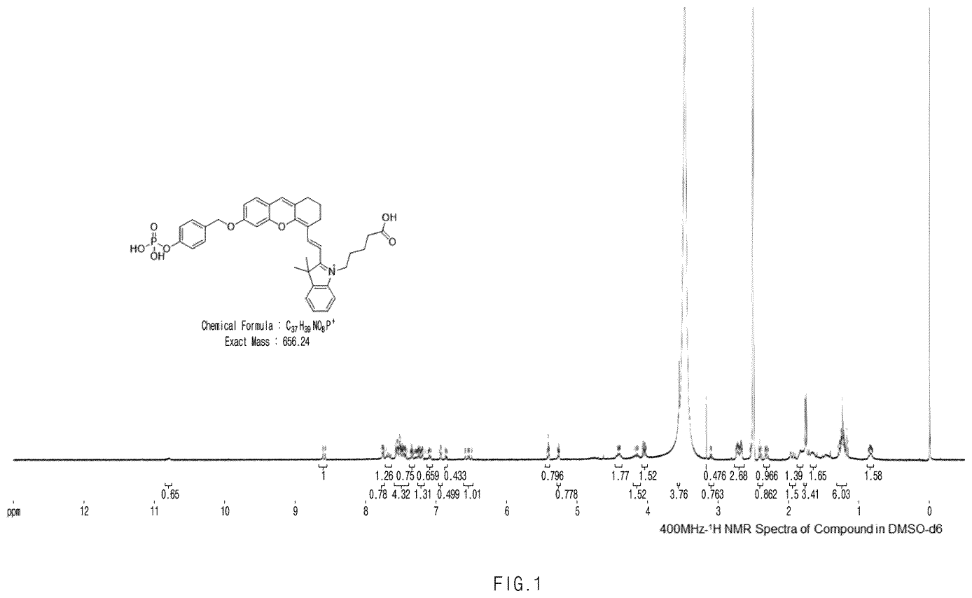

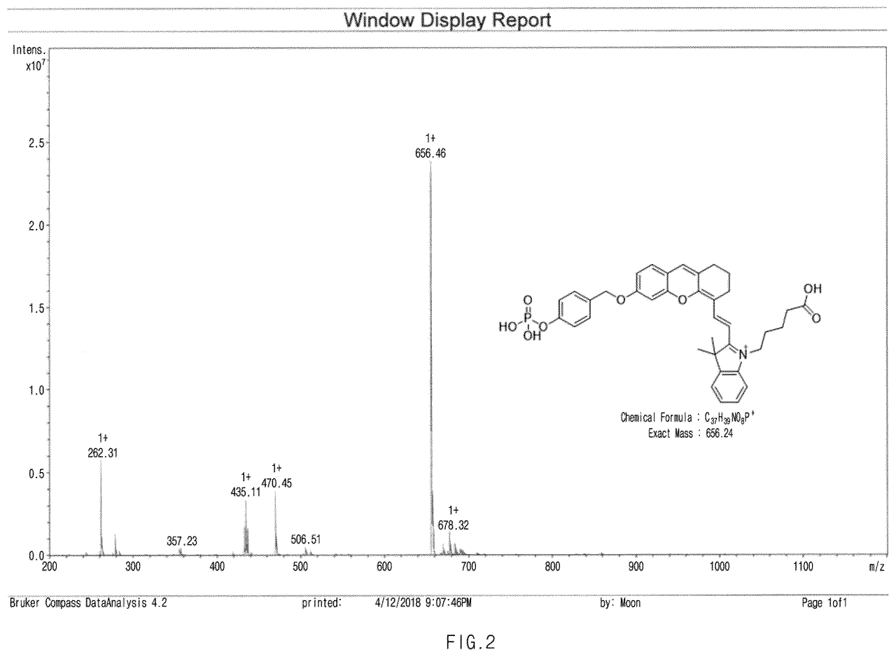

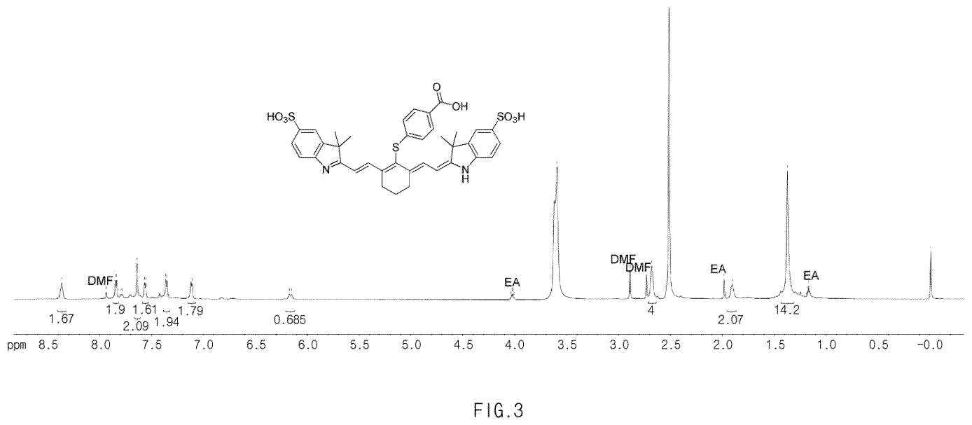

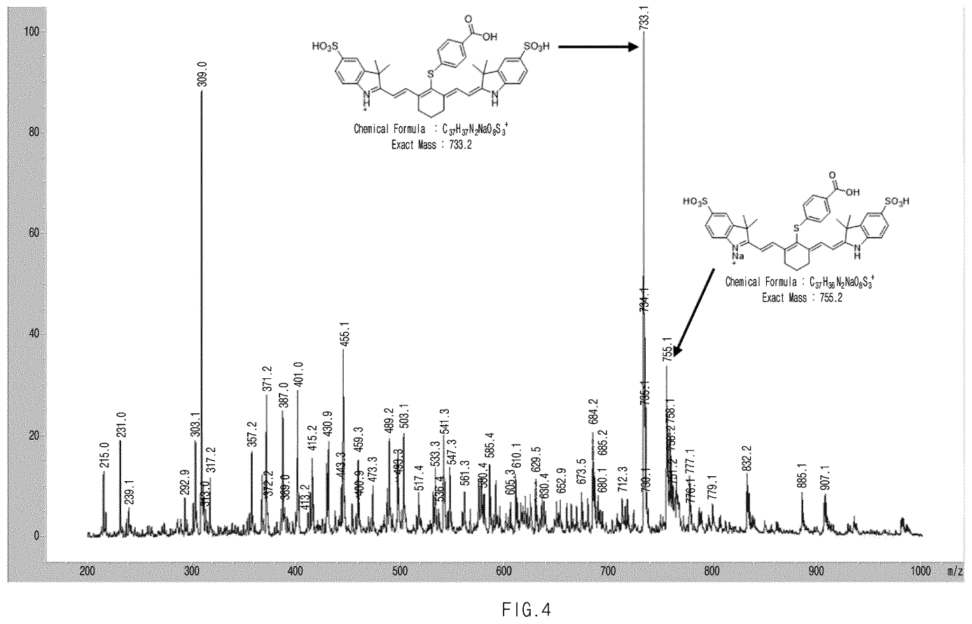

### BEST MODE FOR CARRYING OUT THE INVENTION

Hereinafter, the present invention will be described in detail.

An aspect of the present invention provides a fluorescent probe compound.

Specifically, the fluorescent probe compound may be represented by the following Chemical Formula 1 or Chemical Formula 2.

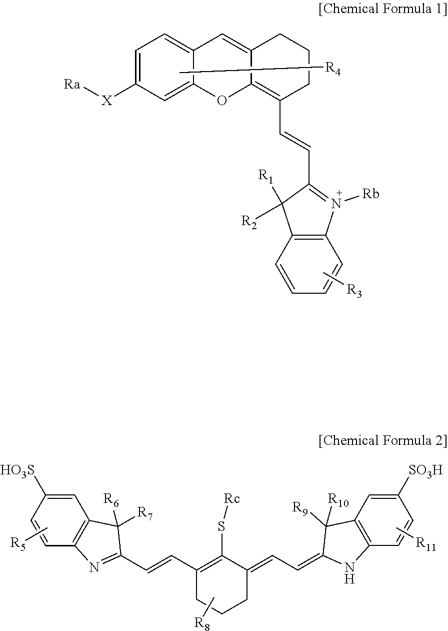

In Chemical Formula 1 and Chemical Formula 2,
X is O or NH,
R₁ to R₁₁ are the same as or different from each other, and are each hydrogen, deuterium, a halogen group, a hydroxy group, an alkoxy group, an amine group, a carboxyl group, an ester group, a substituted or unsubstituted alkyl group, a substituted or unsubstituted alkenyl group, a substituted or unsubstituted alkynyl group, a substituted or unsubstituted cycloalkyl group, a substituted or unsubstituted heterocycloalkyl group, a substituted or unsubstituted aryl group, or a substituted or unsubstituted heteroaryl group, and
Ra is the following Chemical Formula A, Rb is the following Chemical Formula B, and Rc is the following Chemical Formula C.

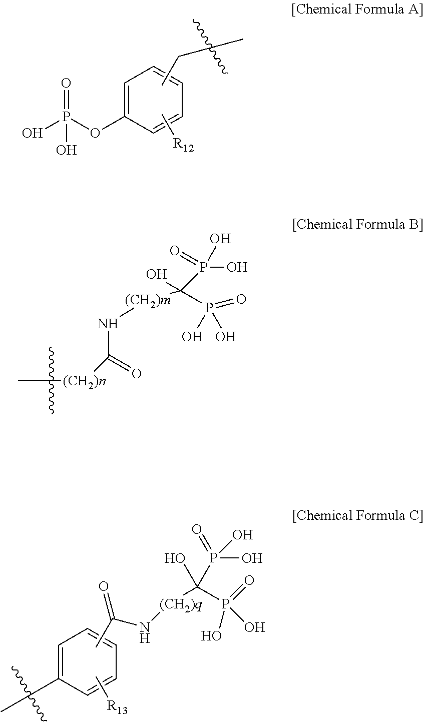

In Chemical Formula A to Chemical Formula C,
R₁₂ and R₁₃ are the same as or different from each other, and are each hydrogen, deuterium, a halogen group, a hydroxy group, an alkoxy group, an amine group, a carboxyl group, an ester group, a substituted or unsubstituted alkyl group, a substituted or unsubstituted alkenyl group, a substituted or unsubstituted alkynyl group, a substituted or unsubstituted cycloalkyl group, a substituted or unsubstituted heterocycloalkyl group, a substituted or unsubstituted aryl group, or a substituted or unsubstituted heteroaryl group, and
n, m, and q are a repeat number of a unit in parentheses, are the same as or different from each other, and are each 0 to 10.

Specifically, in Chemical Formula 1 and Chemical Formula 2, X may be O or NH, and preferably, X may be O.

R₁ to R₁₁ may be the same as or different from each other, and may each be hydrogen, deuterium, a halogen group, a hydroxy group, an amine group, a carboxyl group, a substituted or unsubstituted alkyl group having 1 to 10 carbon atoms, a substituted or unsubstituted cycloalkyl group having 3 to 10 carbon atoms, or a substituted or unsubstituted aryl group having 6 to 20 carbon atoms.

R₁ to R₁₁ may be the same as or different from each other, and may each be hydrogen, a halogen group, a hydroxy group, or a substituted or unsubstituted alkyl group having 1 to 10 carbon atoms.

R₁ to R₁₁ may be the same as or different from each other, and may each be hydrogen or a substituted or unsubstituted alkyl group having 1 to 10 carbon atoms, and more specifically, Ri to R₁₁ may be the same as or different from each other, and may each be hydrogen or a alkyl group having 1 to 10 carbon atoms.

R₁, R₂, R₆, R₇, R₉, and R₁₀ may be the same as or different from each other, and may each be hydrogen, a halogen group, a hydroxy group, or a substituted or unsubstituted alkyl group having 1 to 10 carbon atoms.

R₁, R₂, R₆, R₇, R₉, and R₁₀ may be the same as or different from each other, and may each be hydrogen or a substituted or unsubstituted alkyl group having 1 to 10 carbon atoms, and more specifically, R₁ to R₁₁ may be the same as or different from each other, and may each be hydrogen or an alkyl group having 1 to 10 carbon atoms.

R₁, R₂, R₆, R₇, R₉, and R₁₀ may be the same as or different from each other, and may each be an alkyl group having 1 to 10 carbon atoms.

Specifically, R₁, R₂, R₆, R₇, R₉, and R₁₀ may be the same as or different from each other, and may each be an alkyl group having 1 to 6 carbon atoms, and more specifically, a methyl group, an ethyl group, a propyl group, a butyl group, or a t-butyl group.

R₃, R₄, R₅, R₈, and R₁₁ may be the same as or different from each other, and may each be hydrogen, a halogen group, a hydroxy group, or a substituted or unsubstituted alkyl group having 1 to 10 carbon atoms.

R₃, R₄, R₅, R₈, and R₁₁ may be the same as or different from each other, and may each be hydrogen or a substituted or unsubstituted alkyl group having 1 to 10 carbon atoms, and more specifically, R₁ to R₁₁ may be the same as or different from each other, and may each be hydrogen or an alkyl group having 1 to 10 carbon atoms.

R₃, R₄, R₅, R₈, and R₁₁ may each be hydrogen.

R₁₂ and R₁₃ may be the same as or different from each other, and may each be hydrogen, deuterium, a halogen group, a hydroxy group, an amine group, a carboxyl group, a substituted or unsubstituted alkyl group having 1 to 10 carbon atoms, a substituted or unsubstituted cycloalkyl group having 3 to 10 carbon atoms, or a substituted or unsubstituted aryl group having 6 to 20 carbon atoms.

R₁₂ and R₁₃ may be the same as or different from each other, and may each be hydrogen, a halogen group, a hydroxy group, or a substituted or unsubstituted alkyl group having 1 to 10 carbon atoms.

R₁₂ and R₁₃ may each be hydrogen.

n, m, and q are a repeat number of a unit in parentheses, may be the same as or different from each other, and may be 0 to 10, specifically, 0 to 5, and more specifically, 1 to 4.

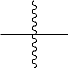

may refer to a binding site.

The halogen group may be a fluorine group (F), a bromine group (Br), a chlorine group (Cl), or an iodine group (I).

The “alkyl group” is an aliphatic hydrocarbon group that does not contain a double bond or a triple bond, which may have 1 to 20, 1 to 19, 1 to 18, 1 to 17, 1 to 16, 1 to 15, 1 to 14, 1 to 13, 1 to 12, 1 to 11, 1 to 10, for example, 1 to 6, particularly, 1 to 4 carbon atoms, and may be linear or branched. Specific examples of the alkyl group include, but are not limited to, a methyl group, an ethyl group, a propyl group, an n-propyl group, an isopropyl group, a butyl group, an n-butyl group, an isobutyl group, a tert-butyl group, a sec-butyl group, a 1-methylbutyl group, a 1-ethylbutyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethylpropyl group, a 1,1-dimethylpropyl group, an isohexyl group, a 4-methylhexyl group, a 5-methylhexyl group, and a benzyl group.

The “alkenyl group” is an aliphatic hydrocarbon group containing at least one double bond, which may have 2 to 20, 2 to 19, 2 to 18, 2 to 17, 2 to 16, 2 to 15, 2 to 14, 2 to 13, 2 to 12, 2 to 11, 2 to 10, for example, 2 to 6, particularly, 2 to 4 carbon atoms, and may be linear or branched. Specific examples of the alkenyl group include, but are not limited to, a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl) vinyl-1-yl group, a 2,2-bis(diphenyl-1-yl) vinyl-1-yl group, a stylbenyl group, and a styrenyl group.

The “alkynyl group” is an aliphatic hydrocarbon group containing at least one triple bond, which may have 2 to 20, 2 to 19, 2 to 18, 2 to 17, 2 to 16, 2 to 15, 2 to 14, 2 to 13, 2 to 12, 2 to 11, 2 to 10, for example, 2 to 6, particularly, 2 to 4 carbon atoms, and may be linear or branched. Specific examples of the alkynyl group include, but are not limited to, an ethynyl group, a propynyl group, a butynyl group, a pentynyl group, and a hexynyl group.

The “cycloalkyl group” is a cyclic aliphatic hydrocarbon group that does not contain a double bond or a triple bond, which may have 3 to 30, 3 to 28, 3 to 26, 3 to 24, 3 to 22, 3 to 20, 3 to 18, 3 to 16, 3 to 14, 3 to 12, 3 to 10, for example, 3 to 8, particularly, 3 to 6 carbon atoms, and may be monocyclic or polycyclic. The polycyclic group refers to a group in which a cycloalkyl group is directly connected or condensed with another ring group, in which the other ring group may be a cycloalkyl group, and may also be another type of ring group, such as a heterocycloalkyl group, an aryl group, or a heteroaryl group. Specific examples of the cycloalkyl group include, but are not limited to, a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, a cyclononyl group, a cyclodecyl group, a cycloundecyl group, a cyclododecyl group, a bicyclo[2.2.1]heptyl group, a bicyclo[2.2.2]octyl group, a bicyclo[3.2.2]nonyl group, a bicyclo[4.4.0]decyl group, and a bicyclo[4.1.0]heptyl group.

The “heterocycloalkyl group” is a cyclic aliphatic hydrocarbon group containing O, S, Se, N, or Si as a heteroatom, which may have 2 to 30, 2 to 28, 2 to 26, 2 to 24, 2 to 22, 2 to 20, 2 to 18, 2 to 16, 2 to 14, 2 to 12, 2 to 10, 2 to 8, 2 to 6, for example, 2 to 5 carbon atoms, and may be monocyclic or polycyclic. The polycyclic group refers to a group in which a heterocycloalkyl group is directly connected or condensed with another ring group, in which the other ring group may be a cycloalkyl group, and may also be another type of ring group, such as a heterocycloalkyl group, an aryl group, or a heteroaryl group. Specific examples of the heterocycloalkyl group include, but are not limited to, aziridine, azetidine, pyrrolidine, piperidine, azepane, azocane, quinuclidine, pyrazolidine, imidazolidine, piperazine (1,2-, 1,3-, and 1,4-isomers), oxirane, oxetane, tetrahydrofuran, oxane (tetrahydropyran), oxepane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, and dithiane.

The “aryl group” is an aromatic hydrocarbon group, which may have 6 to 30, 6 to 28, 6 to 26, 6 to 24, 6 to 22, 6 to 20, 6 to 18, 6 to 16, 6 to 14, for example, 6 to 12 carbon atoms, and may be monocyclic or polycyclic. The polycyclic group refers to a group in which an aryl group is directly connected or condensed with another ring group, in which the other ring group may be an aryl group, and may also be another type of ring group, such as a cycloalkyl group, a heterocycloalkyl group, or a heteroaryl group. In addition, the aryl group includes a spiro group. Specific examples of the aryl group include, but are not limited to, a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, and a condensed ring group thereof.

The “heteroaryl group” is an aromatic hydrocarbon group containing O, S, Se, N, or Si as a heteroatom, which may have 3 to 30, 3 to 28, 3 to 26, 3 to 24, 3 to 22, 3 to 20, 3 to 18, 3 to 16, 3 to 14, 3 to 12, 3 to 10, 3 to 8, 3 to 6, for example, 3 to 5 carbon atoms, and may be monocyclic or polycyclic. The polycyclic group refers to a group in which a heteroaryl group is directly connected or condensed with another ring group, in which the other ring group may be a heteroaryl group, and may also be another type of ring group, such as a cycloalkyl group, a heterocycloalkyl group, or an aryl group. Specific examples of the heteroaryl group include, but are not limited to, a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a qninozolinyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi (dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepine group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, a 5,10-dihydrobenzo[b,e][1,4]azasilinyl, a pyrazolo[1,5-c]quinazolinyl group, a pyrido[1,2-b]indazolyl group, a pyrido[1,2-a]imidazo[1,2-e]indolinyl group, and a 5,11-dihydroindeno[1,2-b]carbazolyl group.

The “alkoxy group” has a chemical structure of —ORₐ, in which Rₐ may be a substituted or unsubstituted alkyl group, and the alkyl group is as described above. Specific examples of the alkoxy group include, but are not limited to, methoxy, ethoxy, n-propoxy, i-propyloxy, n-butoxy, isobutoxy, tert-butoxy, sec-butoxy, n-pentyloxy, neopentyloxy, isopentyloxy, n-hexyloxy, 3,3-dimethyl butyloxy, 2-ethylbutyloxy, n-octyloxy, n-nonyloxy, n-decyloxy, benzyloxy, and p-methylbenzyloxy.

The “amine group” has a chemical structure of —NR_{b}R_{c}, in which R_{b} and R_{c} may each independently be hydrogen, a substituted or unsubstituted alkyl group, a substituted or unsubstituted alkenyl group, a substituted or unsubstituted alkynyl group, a substituted or unsubstituted cycloalkyl group, a substituted or unsubstituted heterocycloalkyl group, a substituted or unsubstituted aryl group, a substituted or unsubstituted heteroaryl group, a hydroxyl group, or a substituted or unsubstituted alkoxy group. The alkyl group, the alkenyl group, the alkynyl group, the cycloalkyl group, the heterocycloalkyl group, the aryl group, the heteroaryl group, and the alkoxy group are as described above.

The “ester group” has a chemical structure of —COOR_{d}, in which R_{d} may be a substituted or unsubstituted alkyl group, a substituted or unsubstituted alkenyl group, a substituted or unsubstituted alkynyl group, a substituted or unsubstituted cycloalkyl group, a substituted or unsubstituted heterocycloalkyl group, a substituted or unsubstituted aryl group, a substituted or unsubstituted heteroaryl group, a hydroxyl group, or a substituted or unsubstituted alkoxy group. The alkyl group, the alkenyl group, the alkynyl group, the cycloalkyl group, the heterocycloalkyl group, the aryl group, the heteroaryl group, and the alkoxy group are as described above.

Meanwhile, the “substitution” referred to in the present invention means that a hydrogen atom in the functional groups and chemical structures described above is replaced with another functional group, such as a halogen atom (F, Br, Cl, or I), a hydroxyl group, a nitro group, a cyano group, an amino group, an azido group, an amidino group, a hydrazino group, a hydrazono group, a carbonyl group, a carbamyl group, a thiol group, a sulfonic acid group, a phosphoric acid group, the above-described alkyl group, the above-described alkenyl group, the above-described alkynyl group, the above-described cycloalkyl group, the above-described heterocycloalkyl group, the above-described aryl group, the above-described heteroaryl group, the above-described alkoxy group, the above-described amine group, a carboxyl group, or the above-described ester group, or a combination thereof, the position to be substituted is not limited as long as it is a position at which the hydrogen atom is substituted, that is, a position at which the substituent may be substituted, and when two or more substituents are substituted, the two or more substituents may be the same as or different from each other.

In particular, Chemical Formula A may be represented by the following Chemical Formula A-1, Chemical Formula B may be represented by the following Chemical Formula B-1, and Chemical Formula C may be represented by the following Chemical Formula C-1.

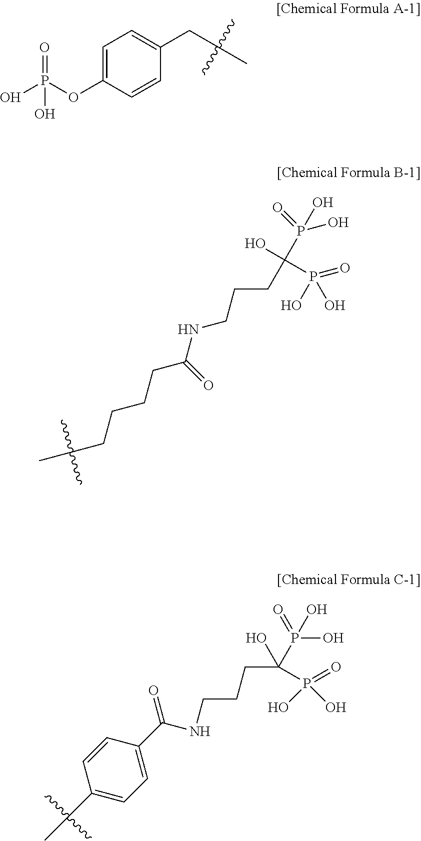 [SMILES: *Cc1ccc(OP(=O)(O)O)cc1 ; *CCCCC(=O)NCCCC(O)(P(=O)(O)O)P(=O)(O)O ; *c1ccc(C(=O)NCCCC(O)(P(=O)(O)O)P(=O)(O)O)cc1]

Chemical Formula 1 may be represented by the following Chemical Formula 1-1. Chemical Formula 2 may be represented by the following Chemical Formula 2-1.

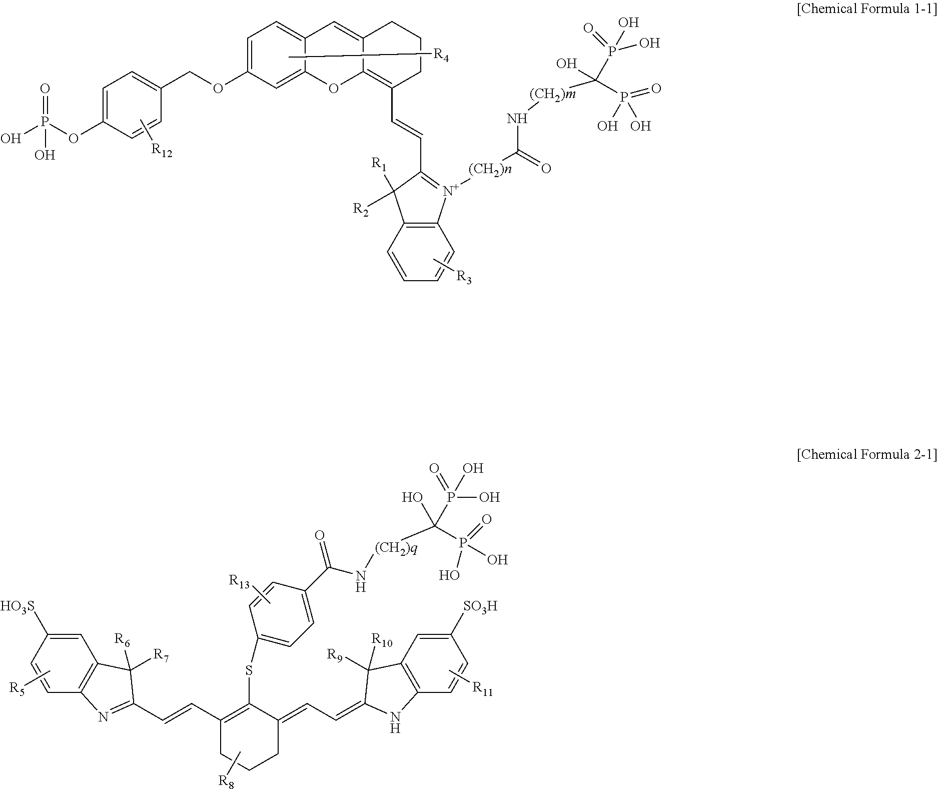

In Chemical Formula 1-1 and Chemical Formula 2-1, the definitions of the respective substituents are as defined in Chemical Formula 1 and Chemical Formula 2.

Chemical Formula 1 may be represented by the following Chemical Formula 1-2.

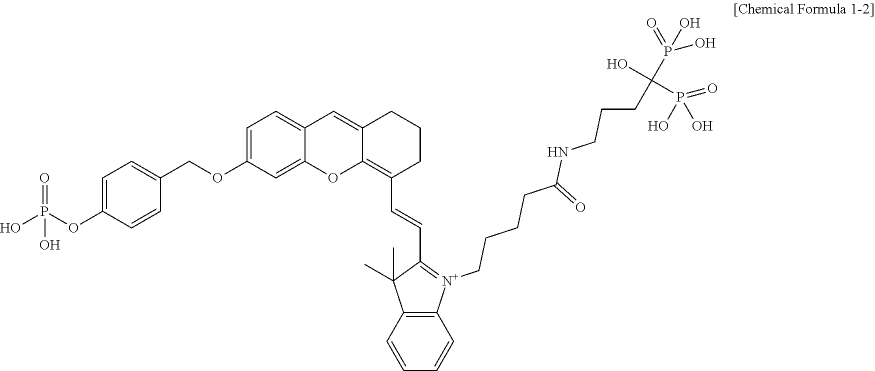 [SMILES: CC1(C)C(/C=C/C2=C3Oc4cc(OCc5ccc(OP(=O)(O)O)cc5)ccc4C=C3CCC2)=[N+](CCCCC(=O)NCCCC(O)(P(=O)(O)O)P(=O)(O)O)c2ccccc21]

Chemical Formula 2 may be represented by the following Chemical Formula 2-2.

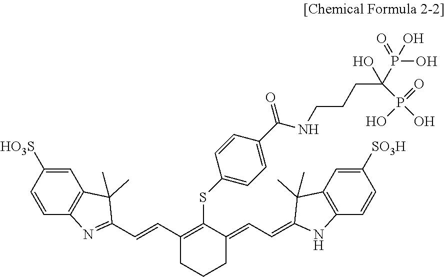 [SMILES: CC1(C)C(/C=C/C2=C(Sc3ccc(C(=O)NCCCC(O)(P(=O)(O)O)P(=O)(O)O)cc3)C(=C/C=C3/Nc4ccc(S(=O)(=O)O)cc4C3(C)C)/CCC2)=Nc2ccc(S(=O)(=O)O)cc21]

The fluorescent probe compound represented by Chemical Formula 1 may be sensitive to a bone-forming activity marker. The bone-forming activity marker may comprise alkaline phosphatase (ALP).

The fluorescent probe compound represented by Chemical Formula 1 may provide a fluorescent signal by undergoing a dephosphorylation reaction through hydrolysis by a bone-forming (osteogenic) activity marker (ALP) due to the excited-state intramolecular proton transfer (ESIP) phenomenon. In addition, the fluorescent probe compound represented by Chemical Formula 1 is near-infrared fluorescence (NIRF) that may exhibit fluorescence in a wavelength range of 650 to 750 nm when injected into a living body, and thus, a bone-forming activity marker may be detected by detecting the fluorescent probe compound.

In addition, the fluorescent probe compound represented by Chemical Formula 1 has a limit of detection (LOD) of 10⁻⁵ to 10⁻³ UmL⁻¹ at which a fluorescence image may be observed through a reaction with the bone-forming activity marker, such that the activity of the bone-forming activity marker of more than 0 UmL⁻¹ and 1.0 UmL⁻¹ or less may be quantified.

The fluorescent probe compound represented by Chemical Formula 2 may be sensitive to a bone-resorbing (osteolytic) activity marker. The bone-resorbing (osteolytic) activity marker may comprise a hydrogen ion (H⁺).

The fluorescent probe compound represented by Chemical Formula 2 may provide a fluorescent signal by undergoing a dephosphorylation reaction through hydrolysis by a bone-resorbing activity marker (hydrogen ion (H⁺)) due to the excited-state intramolecular proton transfer (ESIP) phenomenon. In addition, the fluorescent probe compound represented by Chemical Formula 2 is near-infrared fluorescence (NIRF) that may exhibit fluorescence in a wavelength range of 750 to 850 nm when injected into a living body, and thus, a bone-resorbing activity marker may be detected by detecting the fluorescent probe compound.

In addition, a fluorescence image of the fluorescent probe compound represented by Chemical Formula 2 may be observed through a reaction with a bone-resorbing activity marker, and the activity of the bone-resorbing activity marker may be quantified in a range of pH 1 or higher and pH 6 or lower.

The composite may contain bioceramics; and at least one selected from a fluorescent probe compound represented by Chemical Formula 1 and a fluorescent probe compound represented by Chemical Formula 2 that bind to the bioceramics.

The descriptions of the fluorescent probe compound represented by Chemical Formula 1 and the fluorescent probe compound represented by Chemical Formula 2 may be the same as described above.

The bioceramics may include calcium phosphate-based bioceramics, and specifically, may include at least one selected from hydroxyapatite (HA), α-tricalcium phosphate (α-TCP), and calcium deficient hydroxyapatite (CDHA). In particular, when using a composite in which the fluorescent probe compound described above binds to calcium phosphate-based bioceramics that mimic human bone tissue as described above, there is an advantage of monitoring the metabolic process of osteogenesis and/or osteolysis (resorption) in vivo through near-infrared fluorescence.

The fluorescent probe compound represented by Chemical Formula 1 or the fluorescent probe compound represented by Chemical Formula 2 may be bound by forming an ionic bond with a phosphate group and calcium or a calcium ion of the bioceramics.

In addition, still another aspect of the present invention provides a sensor containing the composite described above.

The sensor refers to a device that measures a physical value and converts the value into a signal that may be read by an observer, and in particular, a biosensor refers to a device in which an observation element is a combination of a physicochemical element and a biological element.

Examples of the sensor include, but are not limited to, a field effect transistor (FET)-based biosensor, a nanoparticle-based biosensor, and a micro electro mechanical system (MEMS)-based biosensor.

In addition, an aspect of the present invention provides a composition for detecting an bone-forming activity marker containing a fluorescent probe compound represented by the following Chemical Formula 1.

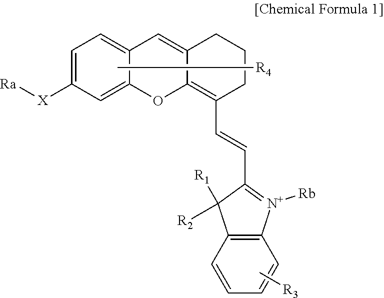

In Chemical Formula 1,
X is O or NH,
R₁ to R₄ are the same as or different from each other, and are each hydrogen, deuterium, a halogen group, a hydroxy group, an amine group, a carboxyl group, a substituted or unsubstituted alkyl group having 1 to 10 carbon atoms, a substituted or unsubstituted cycloalkyl group having 3 to 10 carbon atoms, or a substituted or unsubstituted aryl group having 6 to 20 carbon atoms, and
Ra is the following Chemical Formula A, and Rb is the following Chemical Formula B.

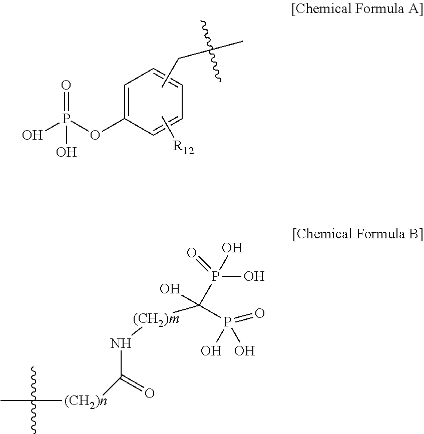

In Chemical Formula A and Chemical Formula B,
R₁₂ is hydrogen, deuterium, a halogen group, a hydroxy group, an amine group, a carboxyl group, a substituted or unsubstituted alkyl group having 1 to 10 carbon atoms, a substituted or unsubstituted cycloalkyl group having 3 to 10 carbon atoms, or a substituted or unsubstituted aryl group having 6 to 20 carbon atoms, and
n and m are a repeat number of a unit in parentheses, are the same as or different from each other, and are each 0 to 10.

The composition for detecting a bone-forming activity marker may contain the fluorescent probe compound represented by Chemical Formula 1 in an amount of 0.1 to 99.9 wt %.

The composition for detecting a bone-forming activity marker may further contain additives such as an excipient, a stabilizer, and a preservative as necessary.

In addition, still another aspect of the present invention provides a composition for detecting a bone-resorbing activity marker containing a fluorescent probe compound represented by the following Chemical Formula 2.

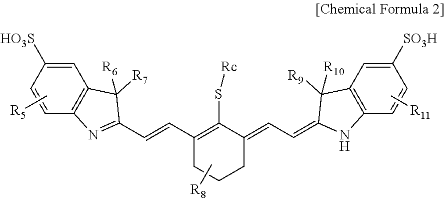

In Chemical Formula 2,
X is O or NH,
R₅ to R₁₁ are the same as or different from each other, and are each hydrogen, deuterium, a halogen group, a hydroxy group, an amine group, a carboxyl group, a substituted or unsubstituted alkyl group having 1 to 10 carbon atoms, a substituted or unsubstituted cycloalkyl group having 3 to 10 carbon atoms, or a substituted or unsubstituted aryl group having 6 to 20 carbon atoms, and
Rc is the following Chemical Formula C.

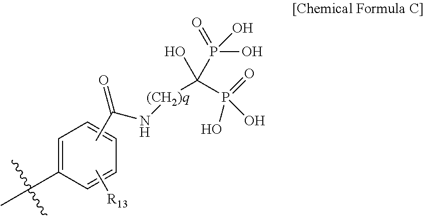

In Chemical Formula C,
R₁₃ is hydrogen, deuterium, a halogen group, a hydroxy group, an amine group, a carboxyl group, a substituted or unsubstituted alkyl group having 1 to 10 carbon atoms, a substituted or unsubstituted cycloalkyl group having 3 to 10 carbon atoms, or a substituted or unsubstituted aryl group having 6 to 20 carbon atoms, and
q is a repeat number of a unit in parentheses and is 0 to 10.

The definitions of the substituents in Chemical Formula 1, Chemical Formula 2, Chemical Formula A, Chemical Formula B, and Chemical Formula C may be the same as described above.

The composition for detecting a bone-resorbing activity marker may contain the fluorescent probe compound represented by Chemical Formula 2 in an amount of 0.1 to 99.9 wt %.

The composition for detecting a bone-resorbing activity marker may further contain additives such as an excipient, a stabilizer, and a preservative as necessary.

Hereinafter, the present invention will be described in more detail with reference to preferred examples.

However, these examples are intended to illustrate the present invention in more detail and are not intended to limit the scope of the present invention.

### <Synthesis Example 1> Synthesis of Bone-Forming Activity Marker-Sensitive Fluorescent Probe Compound

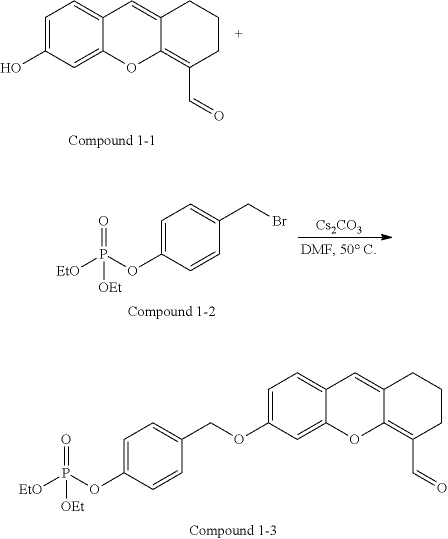

### <1-1> Synthesis of Compound 1-1

Commonly known 6-methoxy-2,3-dihydro-1H-xanthene-4-carbaldehyde was prepared. Then, the 6-methoxy-2,3-dihydro-1H-xanthene-4-carbaldehyde (200 mg, 0.82 mmol) was dissolved in DCM (8.3 mL), and a 1 M BBr₃ DCM solution (20 equivalents, 16.5 mL, 16.5 mmol) was added at 0° C., and stirring was performed at 25° C. for 16 hours. Subsequently, after immersing in a NaHCO₃ solution at 0° C., a water-soluble layer was extracted with DCM/MeOH (10:1 mixed solvent, 2×50 mL). In addition, an organic layer was washed with H₂O and dried with Na₂SO₄. Then, the solvent was removed, and a product was dried in a vacuum to finally obtain a compound 1-1 (6-hydroxy-2,3-dihydro-1H-xanthene-4-carbaldehyde) in the form of a yellow solid (184 mg, 97%).

The ¹H NMR spectrum and mass analysis results of the finally obtained compound 1-1 are shown below.

¹H NMR (DMSO-d6, 600 MHz): δ 10.18 (s, 1H), 7.19 (d, 1H, J=9.0 Hz), 6.92 (s, 1H), 6.61 (sd, 1H, J=2.4 Hz), 6.59 (dd, 1H, J=8.4, 2.4 Hz), 2.53 (t, 2H, J=5.4 Hz), 2.27 (t, 2H, J=5.4 Hz), 1.60 (m, 2H) ppm.

MS (m/z): Calcd, for [MH]+ 229.1, found 229.1.

### <1-2> Synthesis of Compound 1-2

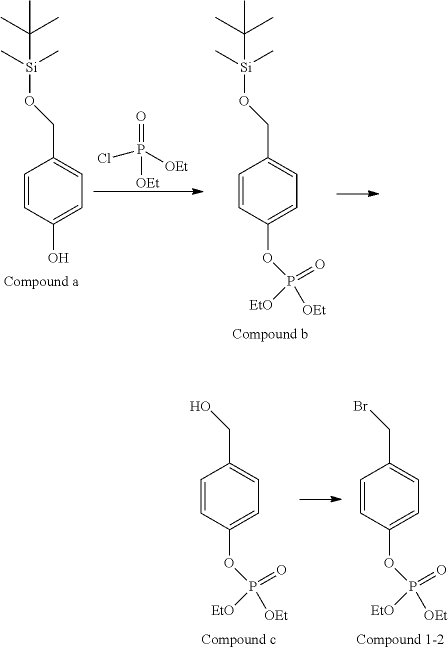

1) Commonly known 4-(((tert-butyldimethylsilyl)oxy)methyl) phenol (compound a) was prepared. In addition, dichloromethane (DCM, 100 mL) was mixed with the previously prepared mixture containing 4-(((tert-butyldimethylsilyl)oxy)methyl) phenol (3.84 g, 16.1 mmol) and trimethylamine (TEA, 11 mL/80.5 mmol), and diethyl chlorophosphate (5.56 g, 32.2 mmol) was added. Subsequently, the reaction mixture was stirred at room temperature in an argon atmosphere overnight.

The reaction mixture was diluted with DCM (100 mL), extracted with water (2×100 mL), and then washed with brine (100 mL). Subsequently, an organic layer was dried with Na₂SO₄ and concentrated under reduced pressure to obtain a crude oil product (compound b). The crude product was used directly in the next process without further purification.

In addition, the previously obtained crude product was stirred with EtOH (100 mL), and then, HCl (8 mL) was added to the reaction mixture at room temperature, stirred for 20 minutes, and neutralized with NaHCO₃. The mixture was extracted with ether (3×80 mL), and an organic layer was dried with Na₂SO₄ and concentrated. Subsequently, purification was performed by silica column chromatography using ethyl acetate/n-hexane (volume ratio: 1/2 to 1/1) as an eluent.

The ¹H NMR, ¹³C{¹H} NMR spectrum and mass spectrometry results of the finally obtained compound c (diethyl(4-(hydroxymethyl)phenyl)phosphate), 3.96 g, 94%) are shown below.

¹H NMR (CDCl₃, 600 MHz): δ 7.32 (d, 2H, J=8.4 Hz), 7.17 (dd, 2H, J=9.0, 1.2 Hz), 4.62 (s, 2H), 4.23 (m, 4H), 1.35 (m, 6H) ppm.

¹³C {¹H} NMR (CDCl₃, 120 MHz): δ 149.90, 149.86, 138.02, 128.26, 119.89, 119.86, 64.66, 64.62, 64.29, 16.08, 16.04 ppm.

MS (m/z): Calcd, for [MH]+ 261.1, found 261.1.

2) Dichloromethane (DCM, 57 mL) was mixed with a mixture containing the prepared compound c (1.2 g, 4.61 mmol) and carbon tetrabromide (2.29 g, 6.92 mmol), and triphenylphosphine (1.81 g, 6.92 mmol) was added at 0° C. Subsequently, the reaction mixture was stirred at room temperature for 7 hours.

The solvent of the reaction mixture was removed by evaporation, and purification was performed by silica column chromatography using ethyl acetate/n-hexane (volume ratio: 1/2 to 1/1) as an eluent.

The ¹H NMR and ¹³C {¹H} NMR spectrum results and the mass spectrometry result of the finally obtained compound 1-2 ((4-(bromomethyl)phenyl diethyl phosphate), 1.3 g, 87%) are shown below.

¹H NMR (CDCl₃, 600 MHz): δ 7.38 (d, 2H, J=9.0 Hz), 7.20 (d, 2H, J=8.4 Hz), 4.48 (s, 2H), 4.24 (m, 4H), 1.37 (m, 6H) ppm.

¹³C{¹H} NMR (CDCl₃, 120 MHz): δ 150.67, 150.63, 134.46, 130.51, 120.30, 120.27, 64.70, 64.65, 32.68, 16.11, 16.07 ppm.

MS (m/z): Calcd, for [MH]+ 323.0, found 323.0.

### <1-3> Synthesis of Compound 1-3

A mixture containing the previously obtained compound 1-1 (278 mg, 1.22 mmol) and compound 1-2 (512 mg, 1.58 mmol), and CS₂CO₃ (1.19 g, 3.66 mmol) was dissolved in anhydrous DMF (6 mL) and stirred at 50° C. for 4 hours. At this time, the stirring was performed in an argon atmosphere.

The solvent of the reaction mixture was removed by evaporation, and purification was performed by silica column chromatography using ethyl acetate/n-hexane (volume ratio: 1/2 to 2/1) as an eluent, thereby finally obtaining a compound 1-3 (diethyl(4-(((4-formyl-2,3-dihydro-1H-xanthen-6-yl)oxy)methyl)phenyl) phosphate). The ¹H NMR spectrum and mass analysis results of the compound 1-3 are shown below.

¹H NMR (CDCl₃, 600 MHz): δ 10.23 (s, 1H), 7.34 (d, 2H, J=9.0 Hz), 7.18 (d, 2H, J=7.8 Hz), 7.02 (d, 1H, J=8.4 Hz), 6.65-6.63 (m, 2H), 6.58 (s, 1H), 4.98 (s, 2H), 4.18 (m, 4H), 2.50 (t, 2H, J=6.0 Hz), 2.38 (t, 2H, J=6.0 Hz), 1.66 (m, 2H), 1.30-1.27 (m, 6H) ppm.

MS (m/z): Calcd, for [MH]+ 471.2, found 471.2.

### <1-4> Synthesis of Compound 1-7

Using the previously obtained compound 1-3, a compound 1-7 was synthesized according to the following reaction scheme.

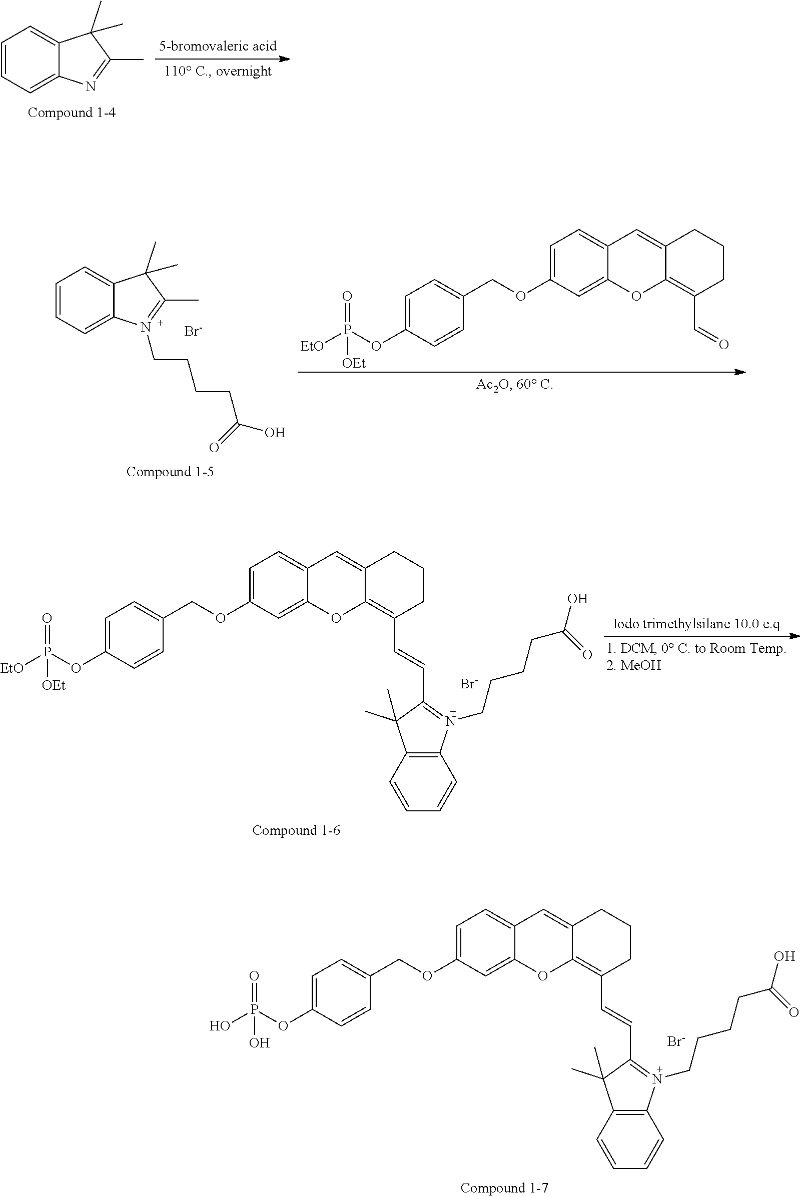

### <Synthesis Example 2> Synthesis of Bone-Resorbing Activity Marker-Sensitive Fluorescent Probe Compound

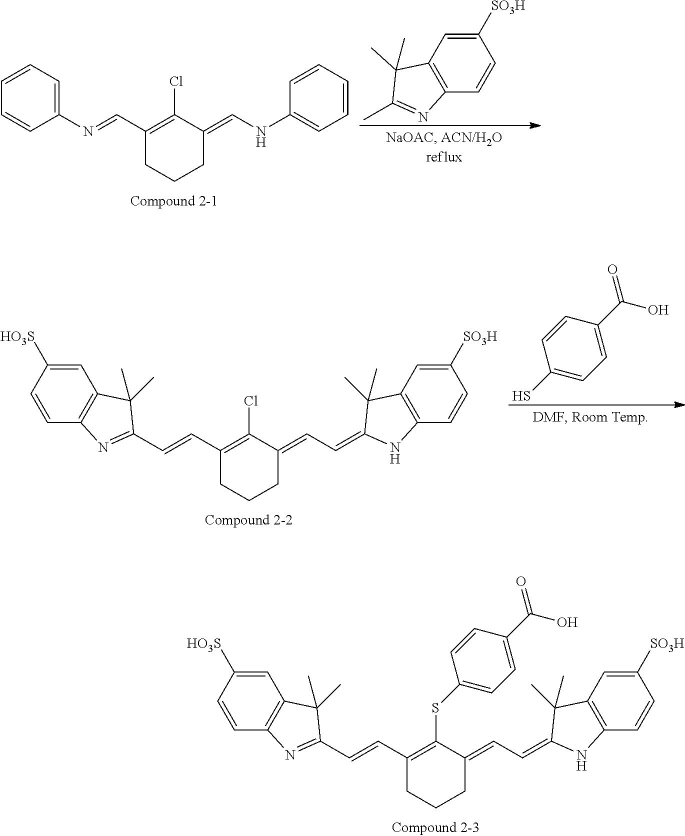

### <2-1> Synthesis of Compound 2-1

At 0° C., phosphorus oxychloride (1.12 g, 12 mmol) was added dropwise to anhydrous DMF (1.37 mL, 17 mmol), and cyclohexanone (0.52 mL, 5.3 mmol) was additionally added after 30 minutes. The mixture prepared as described above was refluxed in a water bath for 1 hour and then cooled to 20° C., and an aniline/ethanol mixture (1:1 (v/v) 18 mL) was added dropwise to the cooled mixture as described above. Then, the mixture was additionally heated for 30 minutes, aniline was added thereto, and the resulting dark purple mixture was poured into cold water/concentrated hydrochloric acid (10:1 (v/v) 11 mL) and left in an ice bath for 2 hours to form crystals. The crystals formed as described above were washed with water and diethyl ether, filtered, and dried to finally obtain a compound 2-1 ((E)-N-(((E)-2-chloro-3-((phenylamino)methylene)cyclohex-1-enyl)methylene)benzeneaminium chloride).

The ¹H NMR spectrum and mass analysis results of the finally obtained compound 2-1 are shown below.

¹H-NMR (DMSO-d⁶, 600 MHz) δ=2.74 (t, 4H), 1.85 (m, 2H), 8.5 (s, 2H), 7.6-7.2 (m, 10H).

MS (m/z): Calcd, for [MH]+ 323.1, found 323.1.

### <2-2> Synthesis of Compound 2-2

1, 2, 3, 3-Tetramethyl-3H-indol-1-ium-5-sulfonate (990 mg), the compound 2-1 (420 mg) obtained above, and sodium acetate (290 mg, 3.54 mmol) were dissolved in ethanol (100 mL), refluxed for 11 hours, and then cooled. Then, 800 mL of ethyl ether (Et₂O) was added dropwise to the cooled mixture, and the formed precipitate was filtered, washed with ethyl ether, and dried in a vacuum overnight to obtain a compound 2-2.

The ¹H NMR spectrum and mass analysis results of the finally obtained compound 2-2 are shown below.

¹H-NMR (DMSO-d⁶, 600 MHz) δ=1.47 (s, 12H), 1.80 (m, 2H), 2.62 (m, 4H), 6.14 (br d, 2H), 7.17 (d, 2H), 7.61 (d, 2H), 7.72 (s, 2H), 8.25 (br d, 2H).

MS (m/z) [MH]+ Calcd, for 643.2, found 643.2.

### <2-3> Synthesis of Compound 2-3

The previously obtained compound 2-2 (500 mg, 0.81 mmol) was dissolved in N, N-dimethylformamide (DMF, 8 mL), 4-mercaptobenzoic acid (138 mg, 0.89 mmol) was added, a reaction was allowed to proceed at room temperature for 15 hours, and then, the solvent was removed. Ethyl acetate (EA) was added to obtain a green solid compound 2-3.

The ¹H NMR spectrum and mass analysis results of the finally obtained compound 2-3 are shown below.

¹H-NMR (DMSO-d⁶, 600 MHz) δ=1.38 (s, 12H), 1.91 (br m, 2H), 2.69 (br m, 4H), 6.16 (d, 1H), 7.12 (d, 2H), 7.36 (d, 2H), 7.56 (d, 2H), 7.65 (s, 2H), 7.79 (d, 2H), 7.84 (d, 2H), 8.73 (br d, 2H).

MS (m/z) [MH]+ Calcd, for 733.2, found 733.1.

### Example 1

The bone-forming activity marker-sensitive fluorescent probe compound (compound 1-7) synthesized in Synthesis Example 1 was bound to calcium-deficient hydroxyapatite (CDHA) as bioceramics to form a composite A. Specifically, as shown in the following reaction scheme, the surface of CDHA (rounded rectangle of the following reaction scheme) was modified with alendronate, and then, the compound 1-7 was reacted to prepare the composite A.

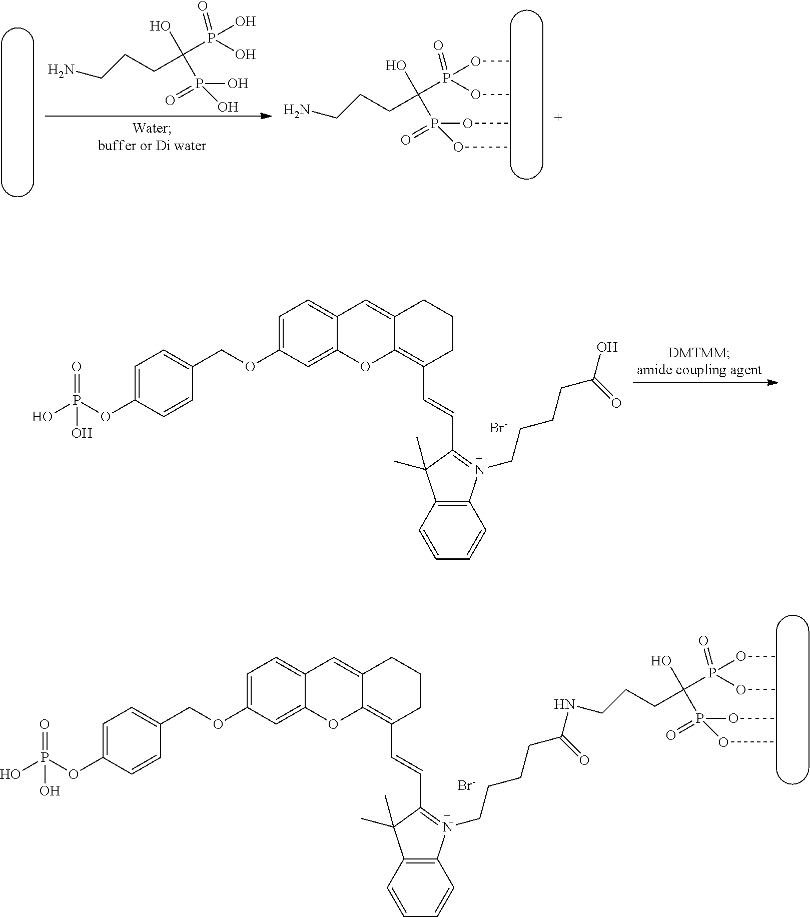

### Example 2

The bone-resorbing activity marker-sensitive fluorescent probe compound (compound 2-3) synthesized in Synthetic Example 2 was bound to CDHA (rounded rectangle of the following reaction scheme) in the same manner as in Example 1, according to the following reaction scheme, to form a composite B.

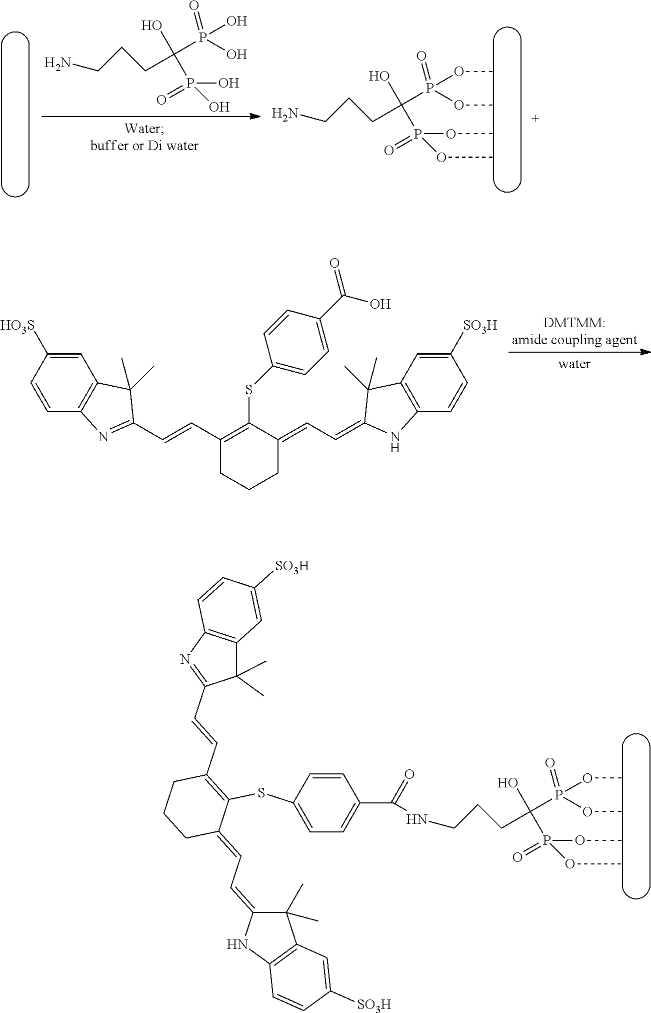

### Example 3

Using a solution obtained by dissolving the bone-forming activity marker-sensitive fluorescent probe compound (compound 1-7) synthesized in Synthesis Example 1 and the bone-resorbing activity marker-sensitive fluorescent probe compound (compound 2-3) synthesized in Synthetic Example 2 at the same concentration, the compound 1-7 and the compound 2-3 were bound to CDHA in the same manner as in Example 1 and Example 2 to form a composite C.

### <Experimental Example 1> Evaluation of Fluorescence of Fluorescent Probe Compounds

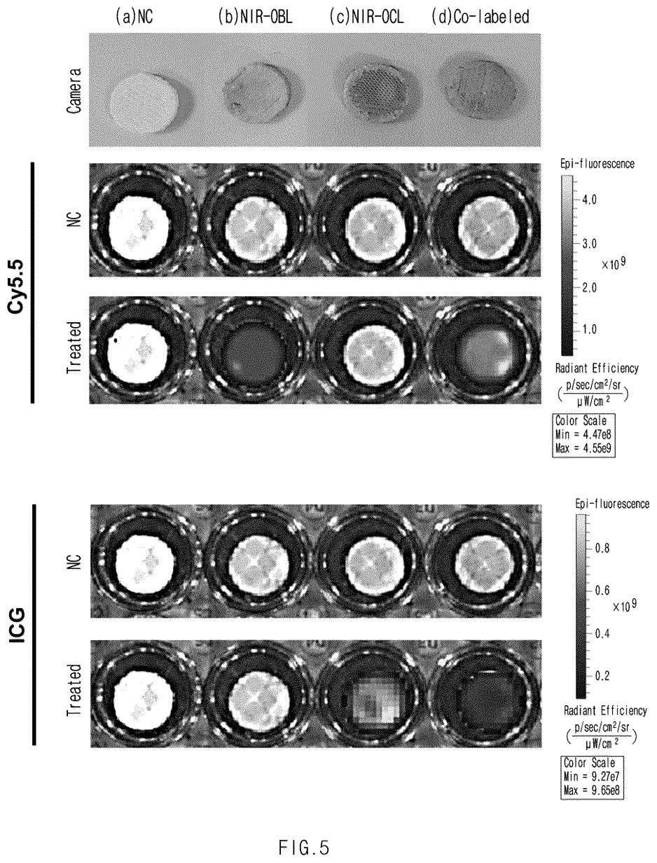

The composite A of Example 1, the composite B of Example 2, the composite C of Example 3, and a negative control (NC) were prepared as CDHA specimens, and the results of examining 1) a fluorescence spectrum that appeared when each specimen was reacted with ALP at 10⁻² U/mL, examining 2) a fluorescence spectrum of each specimen that appeared under conditions of pH 5, and examining 3) a fluorescence image that appeared when each specimen was reacted with ALP at 10⁻² U/mL under conditions of pH 5 were illustrated in FIG. 5. In order to confirm a fluorescence wavelength, Cy5.5 (excited at 675 nm) and ICG (excited at 790 nm) were used as fluorescent dyes.

According to FIG. 5, from the fact that the composite A of Example 1 was reacted with ALP at 10⁻² U/mL and exhibited fluorescence at 675 nm, the composite B of Example 2 exhibited fluorescence at 790 nm at pH 5, and the composite C of Example 3 was reacted with ALP at 10⁻² U/mL at pH 5 and exhibited fluorescence at 675 nm and 790 nm, it was confirmed that the bone-forming activity marker-sensitive fluorescent probe compound of Chemical Formula 1 of the present invention exhibited fluorescence at a wavelength of around 675 nm due to a dephosphorylation reaction by ALP when ALP was present, and it was confirmed that the bone-resorbing activity marker-sensitive fluorescent probe compound of Chemical Formula 2 of the present invention exhibited fluorescence at a wavelength of around 790 nm under conditions of pH 5.

### <Experimental Example 2> Evaluation of Fluorescence According to ALP Concentration and pH Change

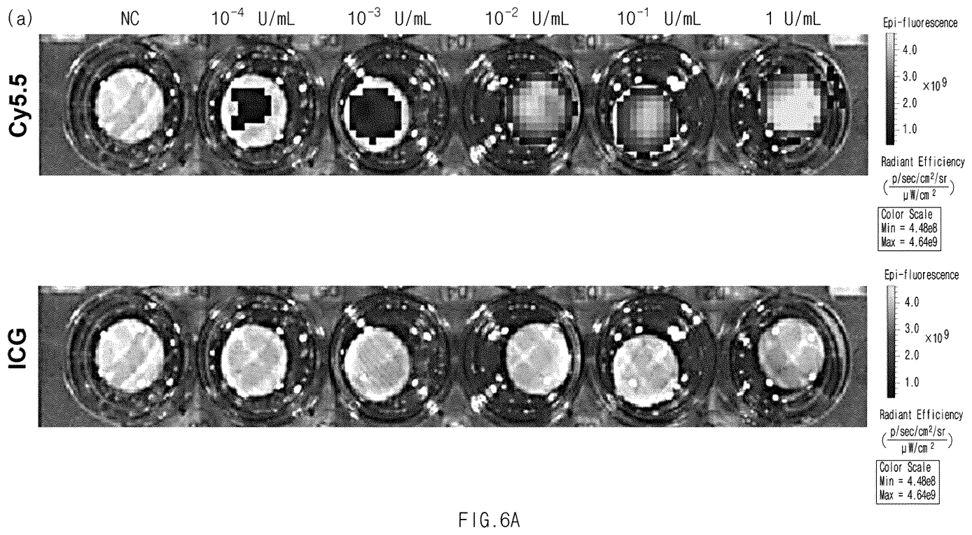

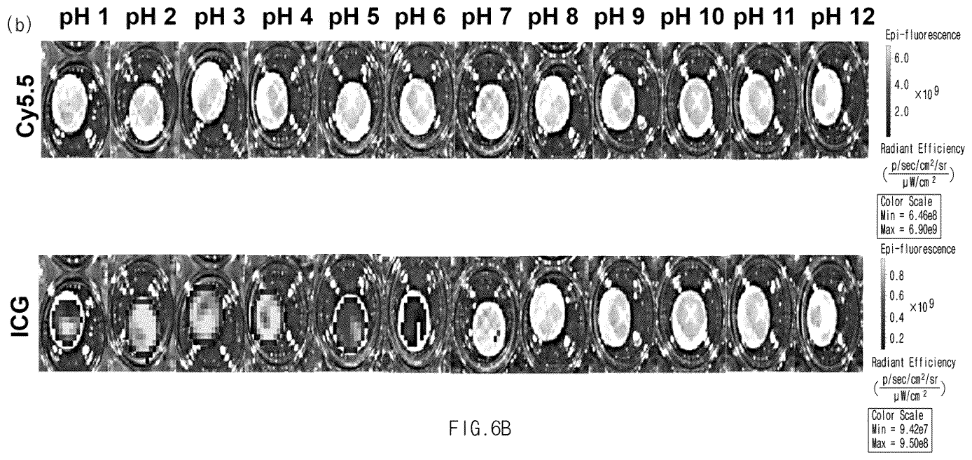

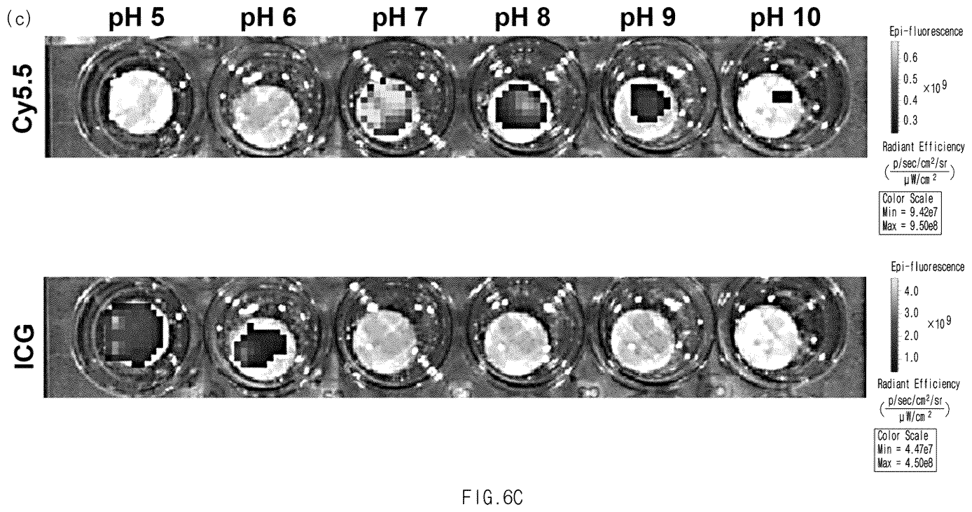

A specimen of the composite A of Example 1 was prepared, and fluorescence images that appeared when reacted with ALP at concentrations of 10⁻⁴ U/mL, 10⁻³ U/mL, 10⁻² U/mL, 10⁻¹ U/mL, and 1 U/mL in a Tri-HCl buffer (10 mM, pH 7.4) were confirmed and illustrated in FIG. 6A. Then, a specimen of the composite B of Example 2 was prepared, and fluorescence images that appeared at pH 1 to pH 12 were confirmed, and a specimen of the composite C of Example 3 was prepared, and fluorescence images that appeared when reacted with ALP at 10⁻² U/mL under conditions of pH 5 to pH 10 were confirmed. The results thereof are illustrated in FIG. 6B and FIG. 6C, respectively.

According to FIG. 6A, it was confirmed that the bone-forming activity marker-sensitive fluorescent probe compound of Chemical Formula 1 of the present invention (composite A) was reacted with ALP at various concentrations (10⁻⁴ to 1 U/mL) and exhibited fluorescence at a wavelength of 675 nm.

In addition, according to FIG. 6B, it was confirmed that the bone-resorbing activity marker-sensitive fluorescent probe compound of Chemical Formula 2 of the present invention (composite B) exhibited fluorescence at a wavelength of 790 nm under conditions of pH 1 or higher and lower than pH 7 (or pH 1 to pH 6).

In addition, according to FIG. 6C, it was confirmed that fluorescence appeared at a wavelength of 790 nm and no fluorescence appeared at a wavelength of 675 nm at lower than pH 7, and it was confirmed that fluorescence appeared at a wavelength of 675 nm and no fluorescence appeared at a wavelength of 790 nm at pH 7 or higher, and thus, it was confirmed that the bone-forming activity marker-sensitive fluorescent probe compound of Chemical Formula 1 of the present invention was reacted with ALP and exhibited fluorescence under conditions of pH 7 or higher.

In addition, in Experimental Example 2, it was confirmed that all fluorescence was exhibited within 10 minutes after the addition of ALP or after the pH change, and thus, it was found that ALP and pH change were monitored significantly quickly.

### <Experimental Example 3> Evaluation of Fluorescence According to Osteoblast and Osteoclast Activity

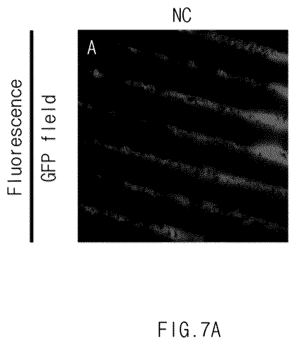

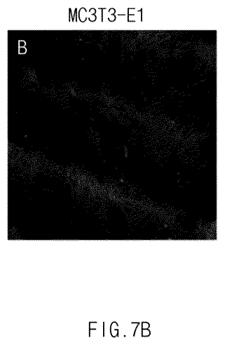

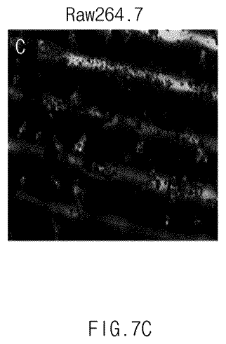

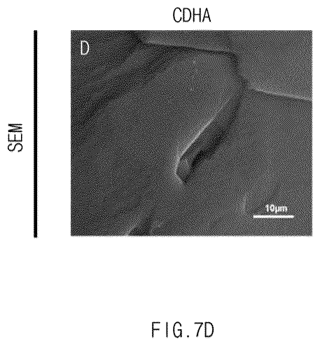

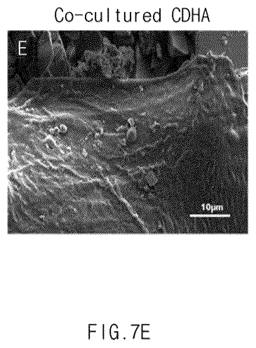

The fluorescence images that appeared after bringing the MC3T3-E1 cell line, which is a precursor cell of an osteoblast, and the Raw264.7 cell line, which is a precursor cell of an osteoclast, into contact with the specimen of the composite C of Example 3 are illustrated in FIGS. 7A to 7E.

FIG. 7A is a negative control in which no treatment is applied to the specimen of the composite C. According to FIG. 7B, it was confirmed that fluorescence appeared due to ALP generated when osteoblasts differentiate by MC3T3-E1, which is a precursor cell of an osteoblast, and according to FIG. 7C, it was confirmed that fluorescence appeared due to pH change generated when osteoclasts differentiate by Raw264.7, which is a precursor cell of an osteoclast.

### <Experimental Example 4> SEM Images of CDHA and Cell-Cultured CDHA

An SEM image of bare CDHA (FIG. 7D) and an SEM image of MC3T3-E1 cells and Raw264.7 cells co-cultured with the composite C of Example 3 (FIG. 7E) are illustrated in FIGS. 7D and 7E.

### <Experimental Example 5> Evaluation of Cytotoxicity According to Concentration of Fluorescent Probe Compound

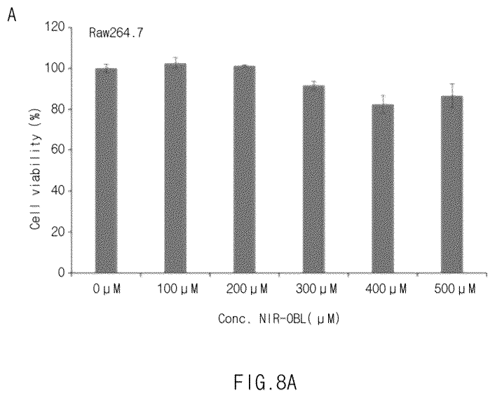

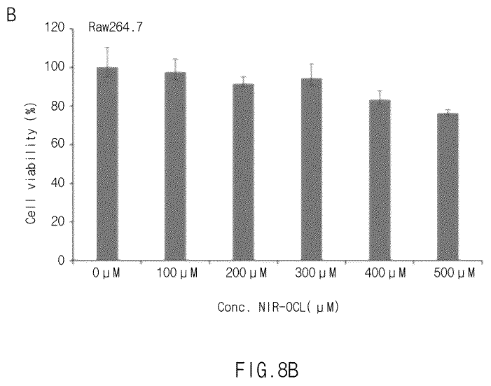

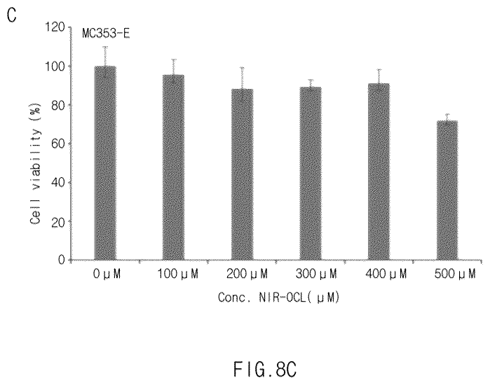

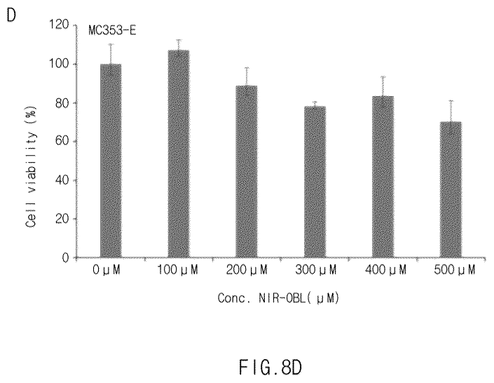

The concentrations of the bone-forming activity marker-sensitive fluorescent probe compound prepared in Synthesis Example 1 and the bone-resorbing activity marker-sensitive fluorescent probe compound prepared in Synthesis Example 2 were set to 0 μM, 100 μM, 200 μM, 300 μM, 400 μM, and 500 μM, respectively, Raw264.7 cells and MC3T3-E1 cells were cultured in a CO₂ 5% incubator for 24 hours, and a cytotoxicity test (CCK-8 kit assay) was performed. The results thereof are illustrated in FIGS. 8A to 8D.

According to FIGS. 8A to 8D, it was confirmed that, even when the concentration of the fluorescent probe compound was 500 μM, the cell viability (%) of each of Raw264.7 cells and MC3T3-E1 cells was 65% or more, which showed that the fluorescent probe compound of the present invention did not exhibit cytotoxicity.

### <Experimental Example 6> Evaluation of Cytotoxicity of Composite

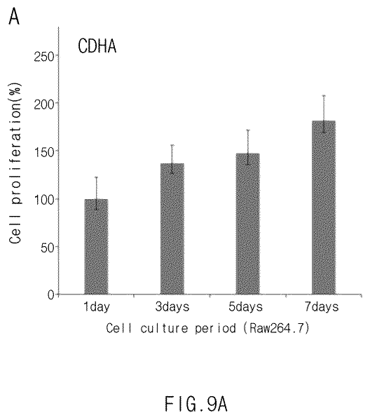

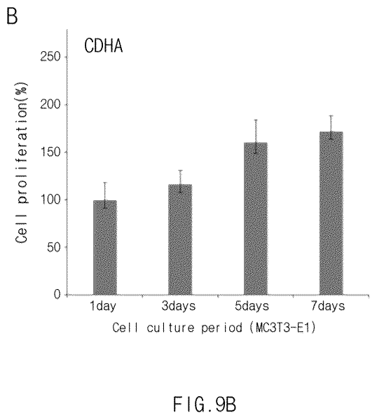

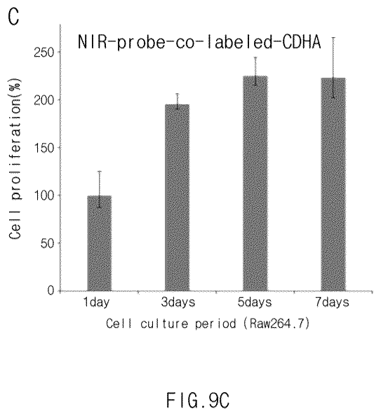

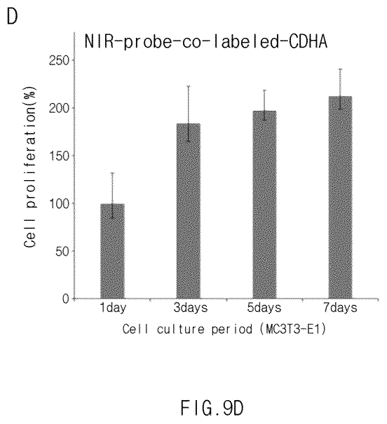

For the bare CDHA not treated with the composite C of Example 3 and the fluorescent probe compound, the cell proliferation (%) of each of Raw264.7 cells and MC3T3-E1 cells on days 1, 3, 5, and 7 in a CO₂ 5% incubator was evaluated using a standard MTT assay. The results thereof are illustrated in FIGS. 9A to 9D.

According to FIGS. 9A to 9D, CDHA used as bioceramics was confirmed to be close to 170% on day 7, confirming that it did not exhibit cytotoxicity (FIGS. 9A and 9B), and the composite C according to the present invention was also confirmed to be close to 200% in the cell proliferation in just 3 days, confirming that it did not exhibit cytotoxicity (FIGS. 9C and 9D).

### <Experimental Example 7> Evaluation of Fluorescence of Fluorescent Probe Compounds According to Osteoblasts/Osteoclasts

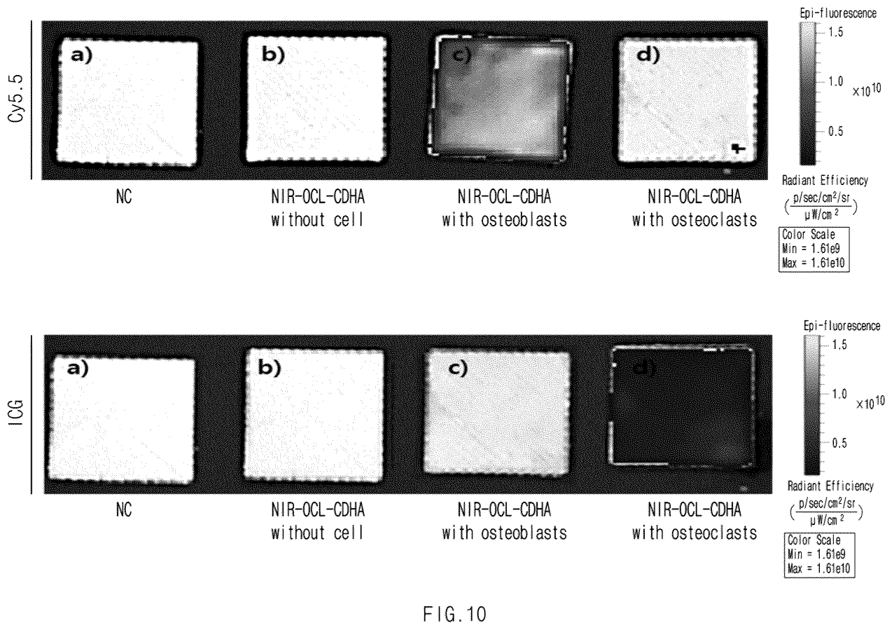

The composite A of Example 1, the composite B of Example 2, and a negative control (NC) were prepared as CDHA specimens, and the results of fluorescence images obtained by examining the CDHA specimens, the composite A without cell treatment, the composite B without cell treatment, the composite A treated with MC3T3-E1 cells, and the composite B treated with Raw264.7 cells were illustrated in FIG. 10.

According to FIG. 10, when the composite A was treated with MC3T3-E1 cells, fluorescence was exhibited at a wavelength of 675 nm, but when the composite B was treated with Raw264.7 cells, fluorescence was not exhibited at a wavelength of 675 nm. In addition, it was confirmed that, when the composite B was treated with Raw264.7 cells, fluorescence was exhibited at a wavelength of 790 nm, but when the composite A was treated with MC3T3-E1 cells, fluorescence was not exhibited at a wavelength of 790 nm.

Therefore, the bone-forming activity marker-sensitive fluorescent probe compound of the present invention was confirmed to react with ALP according to the differentiation of osteoblasts and exhibit fluorescence at a wavelength of 675 nm, and the bone-resorbing activity marker-sensitive fluorescent probe compound of the present invention was confirmed to exhibit fluorescence at a wavelength of 790 nm in response to pH change according to the differentiation of osteoclasts.

### <Experimental Example 8> Evaluation of Dephosphorylation of Fluorescent Probe Compounds

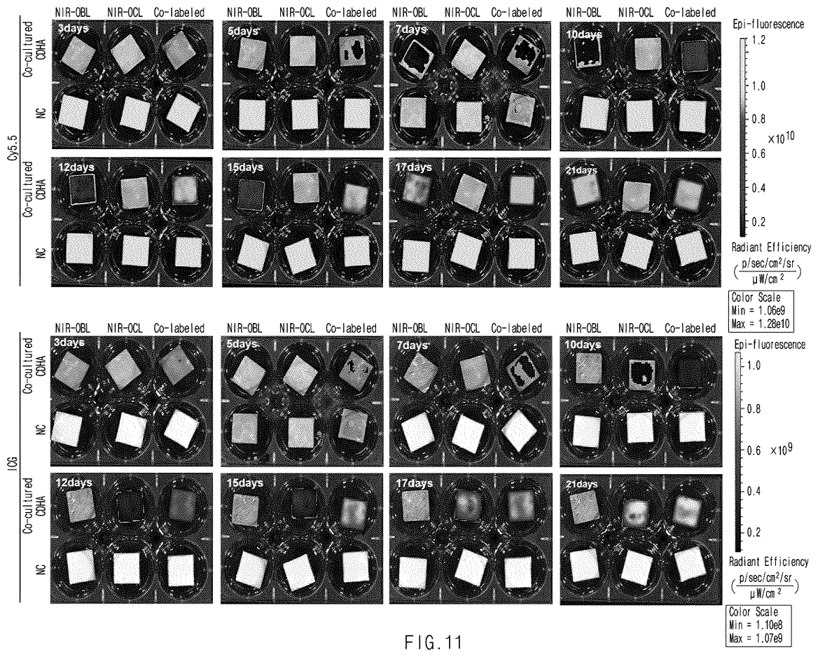

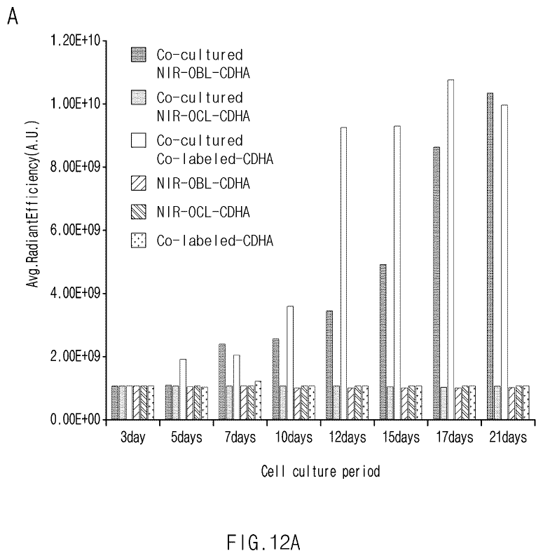

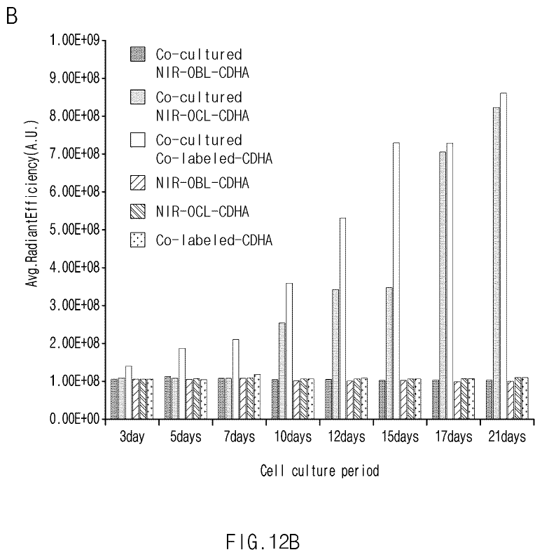

In order to confirm the dephosphorylation of the fluorescent probe compounds by ALP or pH change on CDHA, three bare CDHA specimens and three CDHA specimens cultured with MC3T3-E1 cells and Raw264.7 cells were prepared, the results of examining fluorescence images obtained by treating, the three specimens, one of which was treated with the bone-forming activity marker-sensitive fluorescent probe compound of Synthetic Example 1, another of which was treated with the bone-resorbing activity marker-sensitive fluorescent probe compound of Synthetic Example 2, and the remaining one of which was treated with both the bone-forming activity marker-sensitive fluorescent probe compound and the bone-resorbing activity marker-sensitive fluorescent probe compound, were illustrated in FIG. 11, and the results of measuring fluorescence efficiency (radiant efficiency) in each of six specimens were illustrated in FIGS. 12A and 12B.

According to FIGS. 11 and 12A and 12B, a high fluorescence signal was observed in the group treated with the bone-forming activity marker-sensitive fluorescent probe compound as the diffused ALP hydrolysis increased over time in MC3T3-E1 cells, and a high fluorescence signal was observed in the group treated with the bone-resorbing activity marker-sensitive fluorescent probe compound as the diffused hydrogen ions increased in Raw264.7 cells.

1. A fluorescent probe compound represented by the following Chemical Formula 1 or Chemical Formula 2: 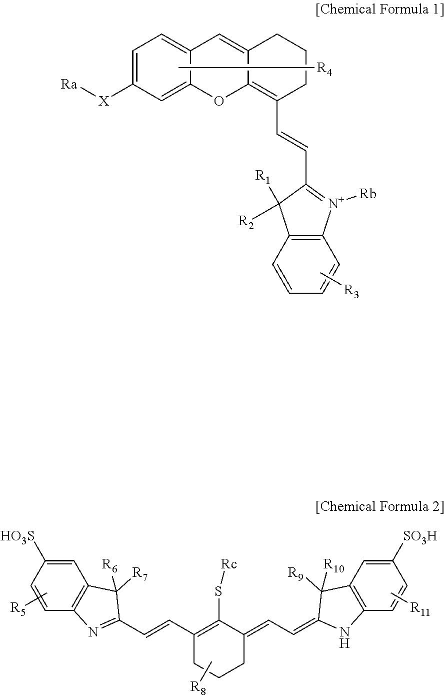
in Chemical Formula I and Chemical Formula 2,
X is O or NH,
R₁ to R₁₁ are the same as or different from each other, and are each hydrogen, deuterium, a halogen group, a hydroxy group, an alkoxy group, an amine group, a carboxyl group, an ester group, a substituted or unsubstituted alkyl group having 1 to 10 carbon atoms, a substituted or unsubstituted cycloalkyl group having 3 to 10 carbon atoms, or a substituted or unsubstituted aryl group having 6 to 20 carbon atoms, and
Ra is the following Chemical Formula A, Rb is the following Chemical Formula B, and Rc is the following Chemical Formula C, 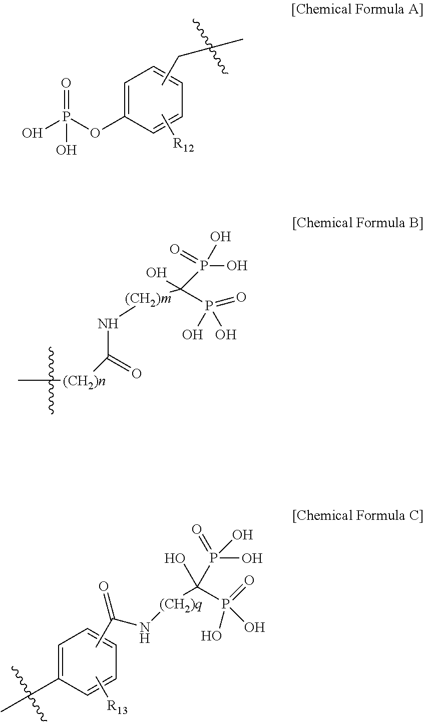
in Chemical Formula A to Chemical Formula C,
R₁₂ and R₁₃ are the same as or different from each other, and are each hydrogen, deuterium, a halogen group, a hydroxy group, an alkoxy group, an amine group, a carboxyl group, an ester group, a substituted or unsubstituted alkyl group having 1 to 10 carbon atoms, a substituted or unsubstituted cycloalkyl group having 3 to 10 carbon atoms, or a substituted or unsubstituted aryl group having 6 to 20 carbon atoms, and
n, m, and q are a repeat number of a unit in parentheses, are the same as or different from each other, and are each 0 to 10.

2. The fluorescent probe compound of claim 1, wherein R₁, R₂, R₆, R₇, R₉, and R₁₀ are the same as or different from each other, and are each an alkyl group having 1 to 10 carbon atoms.

3. The fluorescent probe compound of claim 1, wherein R₃, R₄, R₅, R₈, and R₁₁ are each hydrogen.

4. The fluorescent probe compound of claim 1, wherein n, m, and q are the same as or different from each other and are each 1 to 5.

5. The fluorescent probe compound of claim 1, wherein Chemical Formula 1 is represented by the following Chemical Formula 1-1, and
Chemical Formula 2 is represented by the following Chemical Formula 2-1: 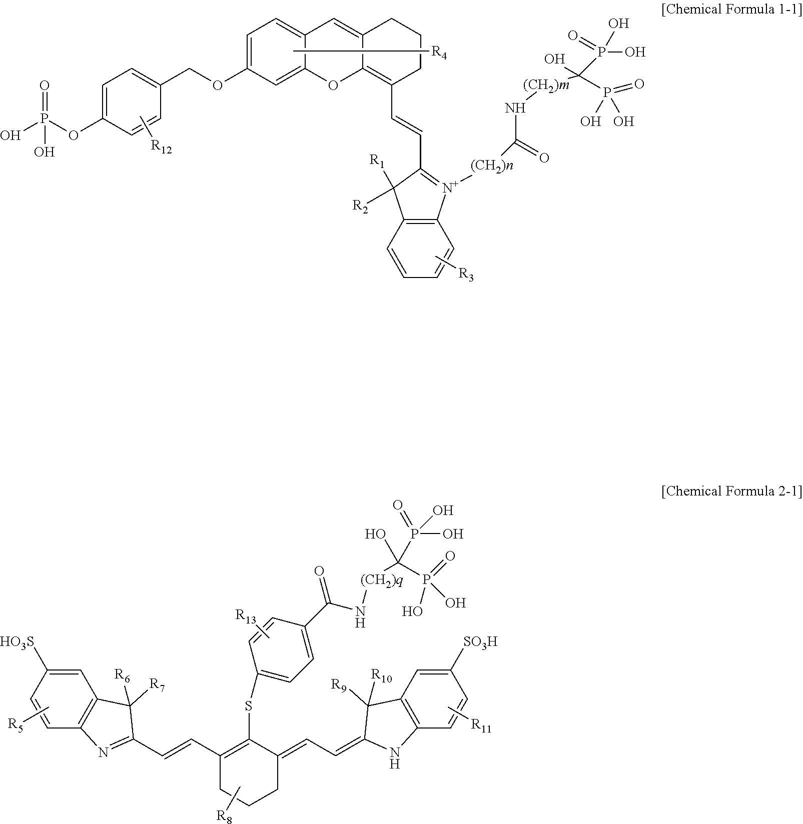
in Chemical Formula 1-1 and Chemical Formula 2-1,
the definitions of substituents are as defined in Chemical Formula 1 and Chemical Formula 2.

6. The fluorescent probe compound of claim 1, wherein Chemical Formula 1 is represented by the following Chemical Formula 1-2, and
Chemical Formula 2 is represented by the following Chemical Formula 2-2:

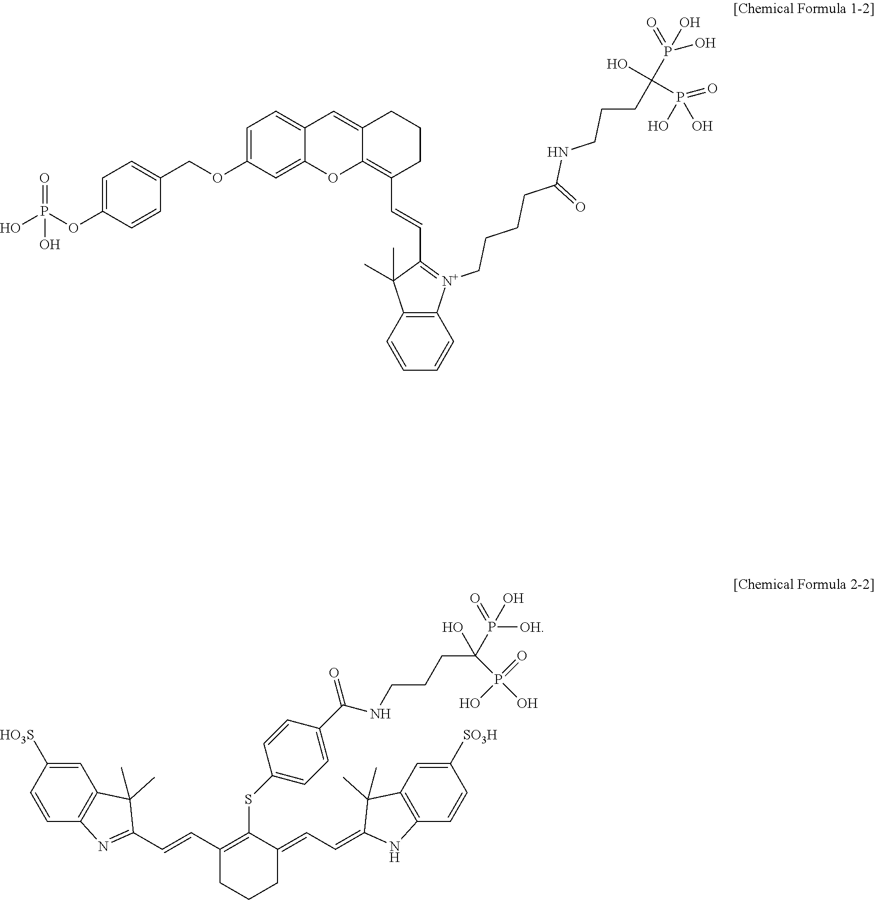 [SMILES: CC1(C)C(/C=C/C2=C3Oc4cc(OCc5ccc(OP(=O)(O)O)cc5)ccc4C=C3CCC2)=[N+](CCCCC(=O)NCCCC(O)(P(=O)(O)O)P(=O)(O)O)c2ccccc21 ; CC1(C)C(/C=C/C2=C(Sc3ccc(C(=O)NCCCC(O)(P(=O)(O)O)P(=O)(O)O)cc3)C(=C/C=C3/Nc4ccc(S(=O)(=O)O)cc4C3(C)C)/CCC2)=Nc2ccc(S(=O)(=O)O)cc21]

7. The fluorescent probe compound of claim 1, wherein the fluorescent probe compound represented by Chemical Formula 1 is sensitive to a bone-forming activity marker, and
the fluorescent probe compound represented by Chemical Formula 2 is sensitive to a bone-resorbing activity marker.

8. The fluorescent probe compound of claim 7, wherein the bone-forming activity marker comprises alkaline phosphatase (ALP), and
the bone-resorbing activity marker comprises a hydrogen ion (H⁺).

9. A composite comprising:
bioceramics; and
at least one fluorescent probe compound selected from the group consisting of a compound represented by the following Chemical Formula 1 and a compound represented by the following Chemical Formula 2, wherein the compound is bound to the bioceramics, 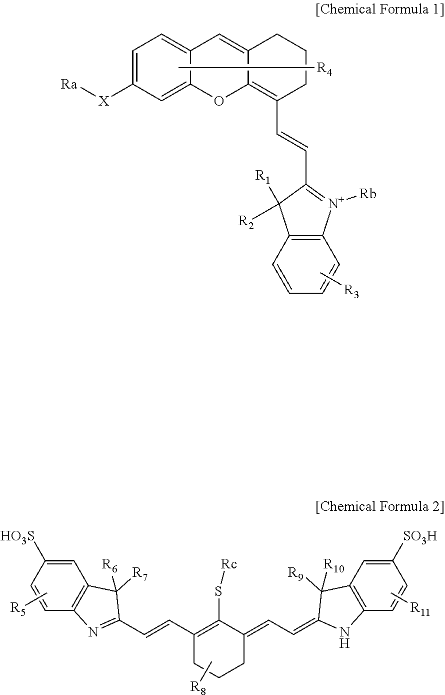
in Chemical Formula 1 and Chemical Formula 2,
X is O or NH,
R₁ to R₁₁ are the same as or different from each other, and are each hydrogen, deuterium, a halogen group, a hydroxy group, an alkoxy group, an amine group, a carboxyl group, an ester group, a substituted or unsubstituted alkyl group having 1 to 10 carbon atoms, a substituted or unsubstituted cycloalkyl group having 3 to 10 carbon atoms, or a substituted or unsubstituted aryl group having 6 to 20 carbon atoms, and
Ra is the following Chemical Formula A, Rb is the following Chemical Formula B, and Rc is the following Chemical Formula C, 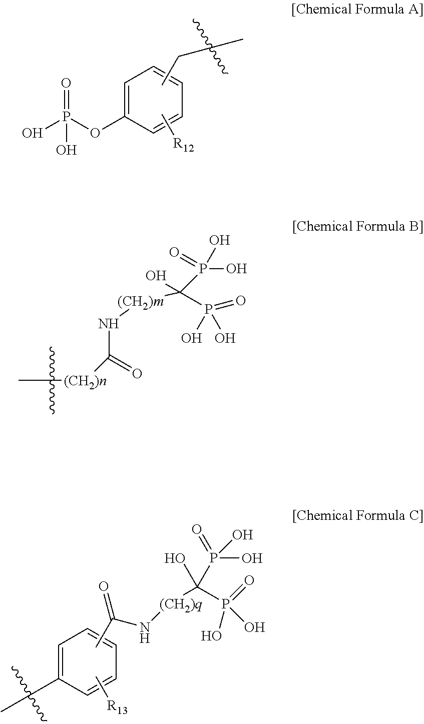
in Chemical Formula A to Chemical Formula C,
R₁₂ and R₁₃ are the same as or different from each other, and are each hydrogen, deuterium, a halogen group, a hydroxy group, an alkoxy group, an amine group, a carboxyl group, an ester group, a substituted or unsubstituted alkyl group having 1 to 10 carbon atoms, a substituted or unsubstituted cycloalkyl group having 3 to 10 carbon atoms, or a substituted or unsubstituted aryl group having 6 to 20 carbon atoms, and
n, m, and q are a repeat number of a unit in parentheses, are the same as or different from each other, and are each 0 to 10.

10. The composite of claim 9, wherein the bioceramics comprise at least one selected from hydroxyapatite (HA), α-tricalcium phosphate (α-TCP), and calcium deficient hydroxyapatite (CDHA).

11. A sensor comprising the composite of claim 9.

12. A method for detecting a bone-forming activity marker in a subject in need thereof, comprising administering the fluorescent probe compound represented by Chemical Formula 1 of claim 1 to the subject.

13. A method for detecting a bone-resorbing activity marker in a subject in need thereof, comprising the fluorescent probe compound represented by Chemical Formula 2 of claim 1 to the subject.

14. The method of claim 12, wherein Chemical Formula 1 is represented by the following Chemical Formula 1-2:

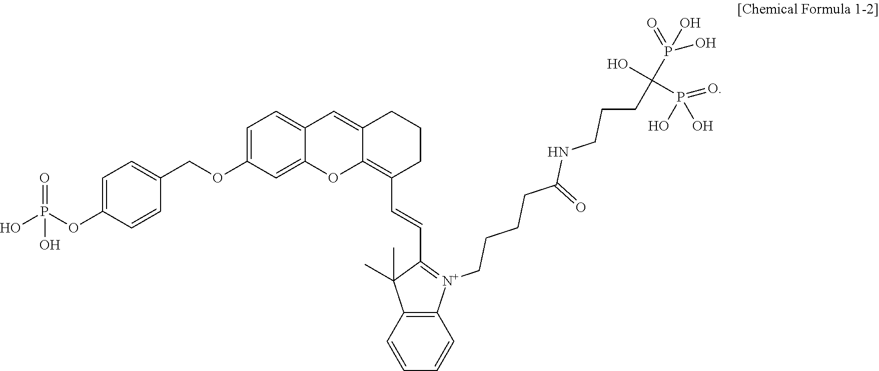 [SMILES: CC1(C)C(/C=C/C2=C3Oc4cc(OCc5ccc(OP(=O)(O)O)cc5)ccc4C=C3CCC2)=[N+](CCCCC(=O)NCCCC(O)(P(=O)(O)O)P(=O)(O)O)c2ccccc21]

15. The method of claim 13, wherein Chemical Formula 2 is represented by the following Chemical Formula 2-2: 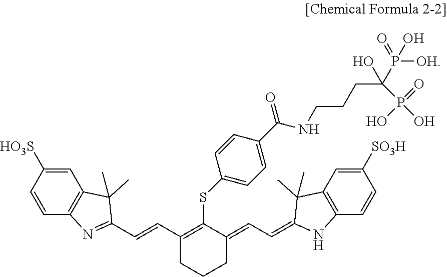 [SMILES: CC1(C)C(/C=C/C2=C(Sc3ccc(C(=O)NCCCC(O)(P(=O)(O)O)P(=O)(O)O)cc3)C(=C/C=C3/Nc4ccc(S(=O)(=O)O)cc4C3(C)C)/CCC2)=Nc2ccc(S(=O)(=O)O)cc21]
